(19) United States
(12) Patent Application Publication
(10) Pub. No.: US 2009/0081775 A1
(43) Pub. Date: Mar. 26, 2009

(54) Microscope system and screening method for drugs, physical therapies and biohazards

(75) Inventors: Erlend Hodneland, Bergen (NO); Hans-Hermann Gerdes, Bergen (NO)

Correspondence Address: BIRCH STEWART KOLASCH & BIRCH, PO BOX 747, FALLS CHURCH, VA, 22040-0747, US

(73) Assignee: Stiftesen Unversitetsforskning Bergen, Bergen (NO)

(21) Appl. No.: 11/920,926

(22) PCT Filed: May 26, 2006
(86) PCT No.: PCT/EP2006/005084

§ 371 Date: Mar. 10, 2008

(30) Foreign Application Priority Data
May 25, 2005 (EP) 05011385.1

(51) Int. Cl. G06K 9/00; C12N 1/00

(52) U.S. Cl. 435/317.1; 382/133

(57) ABSTRACT

Method and device for automated cell analysis and determination of transport and communication between living cells by analyzing the formation of tunneling nanotubes (TNTs) between cells. This method comprising the steps of singularizing cells in a culture medium and staining the cells with a fluorescent or luminescent dyes for staining of cytoplasm and membranes as well as TNTs, flagella and other cell particles for 3-D cell microscopy. The method comprises further an image analysis system.

### FIELD OF THE INVENTION

The present invention relates to method for identification of tunneling nanotubes (TNTS) in 3-D fluorescent images, and in particular to a method for screening of drugs and bioeffective electromagnetic radiation.

### BACKGROUND OF THE INVENTION

Recently we discovered a new biological principle of cell-to-cell communication which is based on nanotubular structures (TNTs) formed de novo between cells (EP-A-1 454 136; Rustom et al., Science 2004; 303:1007-1010). TNTs are structured as thin tubes (50-200 nm in diameter) crossing from one cell to another cell at their nearest distance so that in microscopic images they are seen as straight lines between living cells. They facilitate the selective intercellular transfer of membrane vesicles, organelles, plasma membrane components, cytoplasm, calcium ions and presumably genetic material. Because TNTs seem to be a general phenomenon, assignable to many if not all cell-types, the discovery of these conspicuous structures forced to reconsider all previous conceptions of intercellular communication. In this respect, very recent investigations showed that TNTs are fulfilling essential tasks during the development and maintenance of multicellular organisms, e.g. in the immunsystem, where they mediate the transfer of MHC molecules (Onfelt et al., J. Immunol. 2004, 173, 1511-1513) and calcium ions at the immunological synapse (Watkins et al., Immunity 2005, 23, 309-18). We have also shown that the tunneling nanotubes (TNTs) provide the structural basis for a new type of cell-to-cell communication. TNTs also appear in fixed cells, but they exhibit extreme sensitivity and they are easily destroyed as e.g. prolonged light excitation leads to visible vibrations and rupture. Thus, not only bioactive substances such as drugs but also electromagnetic fields (EMF) such as light and microwaves may compromise TNT-dependent cell-to-cell communication and cause pathological effects in multicellular organisms. However, there are no analyses tools available nor a method for determining the biological effect of a bioactive substance or EMF on the TNT-dependent cell to cell transport and communication.

As a consequence of the important physiological functions of TNTs as well as their predicted link to a great variety of diseases, like e.g. cancer (Vidulescu et al. J. Cell. Mol. Med. 2004, 36, 319), there is a demand for a novel drug screening system providing a system to quickly screen at a large scale a great variety of chemical compounds on their influence on TNTs and TNT-based cellular networks. Therefore, a selective manipulation of TNTs may represent an important new tool for many kinds of therapeutic approaches. In other words, there is demand for a method for quickly testing and screening for a great variety of chemical compounds and their influence on TNTs.

### SUMMARY OF THE INVENTION

Here we propose to use natural nanotubes as sensors for electromagnetic pollution in order to evaluate both the beneficial and negative effects of drugs and electromagnetic field exposure. To further explore and measure these effects, automated detection and quantification are provided. Our approach for identification and quantification of TNTs and TNT development are based on a combination of known image processing techniques and biological cell markers. Watershed segmentation, edge detectors, and optionally, ridge enhancement are used to find TNTs, and image artifacts. Mathematical morphology is employed at several stages of the processing chain for measuring these effects.

Consequently, a method for automated cell analysis, cell classification and/or determination of transport and communication between living cells is provided, comprising the steps of singularizing cells in a culture medium and spreading or plating cells in a monolayer onto a substrate for a predetermined period; staining the cells with a fluorescent or luminescent dye, immunofluorescence or other detectable microscopic stain to obtain stained plasma membranes, TNTs, flagella and/or other cell particles for 3-D cell microscopy; performing image acquisition in multiple focal planes; analysing the images of the multiple focal planes as to the staining intensity over background in predetermined volumes to obtain stained 2-D and 3-D structures; segmenting structures into regions and classifying the regions as to shape, curvature and other selected properties; selecting structures that are candidates for TNTs or flagellae based on the property that a TNT or a flagella must cross background; reducing the number of candidates for TNTs or flagellae by keeping or, in the case of flagellae, rejecting those crossing from one cell to another. In a preferred embodiment of the invention, a ridge enhancing curvature depending filter is applied to the surface stained images to enhance plasma membranes. As an alternative, it is also possible to apply a ridge enhancement to the image which is then followed by an adaptive thresholding. The ridge enhancement is described in detail below and enhances the ridges of the image, which includes both the cell border and the TNTs. With the method of the invention, organelle transport between cells is preferably investigated. A further important aspect of the invention is the automated, and thus more objective, investigation of semen quality and other structure comprising tube or flagellae like extensions.

A preferred embodiment of the method of the invention comprises the use of a substrate that has been coated to obtain a microarray of essentially singularised cells having predetermined distances to each other. When cells are plated on such type of substrates the image analysis becomes easier and more reliable. This preferred embodiment is achieved by plating cells on a substrate which bears a patterned coating (lines, circles, waves), e.g. applied by photolithography.

A further embodiment of the invention comprises the addition of a chemical compound, a therapeutic substance, a medicament or a suspected pharmaceutically effective substance to the culture medium. Physical effects on cells can further be investigated according to the invention. In this case, the cells in the culture medium are subjected to physical effects such as heat, radiation, mechanical stress, and electromagnetic fields for a predetermined period of time. These physical effects can come from potential biohazards or from therapeutic devices.

The microscope set-up in accordance with the invention comprises a 3-D microscope, a Z-stepper, and an image acquisition and analysis system for automated cell analysis, cell classification and/or determination of transport and communication between cells, and optionally, a micropatterned substrate for plating an array of cells having essentially uniform distances to each other. This device or system may be used for serial investigation of the quality of semen and suspected pharmaceuticals and active mediums, particularly, for the treatment of tumors, of high blood pressure, of viral, bacterial or parasitic infection diseases, disorders of the metabolism, disorders of the nervous system, the psyche or the mind, and of the cholesterol level. Another aspect of the invention relates to the investigation of effective substances in gene therapy, for cell targeting and in pharmacology.

A further aspect of the invention relates to a procedure and a device for a quantitative analysis of TNT-rupture by drugs, heat and electromagnetic fields. As mentioned above, in an embodiment of the invention the cell cultures for the development of TNTs are grown on micropatterned surfaces to obtain standard cell growth and more uniform TNTs for automated analysis. Such a system stands out by an innovative cell culture system, allowing controlled and reproducible cell growth as well as a fully computerised analysis system, ensuring an unbiased and fast data processing. Furthermore, a process is provided for the automated quantification of the number of TNTs in the acquired image stacks. A further aspect of the invention relates to a set-up for performing quantitative measurements (microscope set-up, software package, micropatterned dishes for standardized cell growth and TNT development, and, optionally, EMF generator) which can be employed by manufactures and institutions wishing to assess the biological effects of electromagnetic fields, for example, the pharmaceutical and medical field, manufactures of mobile phones, research institutes assessing environmental pollution.

Another aspect of the invention relates to a screening system which comprises three main components. The first is a specialized cell culture system providing reproducible and optimised growth conditions essential for TNT analysis. The cell culture system makes use of chemically functionalised glass surfaces. These surfaces allow to grow cells in a predefined pattern, i.e. with an optimal distance for TNT formation as well as minimized cell clustering, thus, leading to a maximal reproducibility of the following steps of analysis. After application of pharmaceuticals, surfaces will be analysed by a specialized “high throughput” microscope, the second component. This microscope system captures automatically a defined number of 3D stacks in random areas of the respective surfaces. For this purpose, the microscope is equipped with an autofocus function, a programmable, motor-driven dish holder and an appropriate control software. Comparable microscopic systems are already available from some microscope distributors. The third part of the screening system is a specialised, fully automated method, which analyses the acquired 3D image data by detecting and counting TNTs between the cells as well as quantifying the amount of TNT-dependent, intercellular organelle transfer. By a combination of the three main components, the drug screening system provides a set-up allowing an unbiased, reproducible and fast processing of TNTs related topics.

The complete system offers pharmaceutical companies an ideal set-up to screen on a large scale for chemical compounds selectively affecting TNT formation, TNT stability as well as TNT mediated organelle transfer. With respect to the important functions of TNTs, such chemicals could have an immense value for future pharmaceutical developments. The chemically functionalised glass surfaces can be optimised and adopted for many different cell-systems, thus providing ideal platforms, whenever a reproducible, controlled cell growth is desired, e.g. during all aspects of tissue engineering. This offers new perspectives for industry as well as basic research. The optimized “high throughput” microscope in combination with the automated method for TNT analysis represents an interesting, highly flexible imaging system, which can easily be adapted to various scientific questions.

In this respect the drug screening system according to the invention provides the first and sole system to analyse for TNT-based cell interactions and can be in particular used in the medical research on the treatment of a great variety of diseases, such as cancer, diabetes, high blood pressure, etc. Of great value are also chemically functionalised glass/dish surfaces allowing pattern-controlled cell growth. Such devices are also of interest for applications reaching from tissue engineering to basic research.

Automated methods for identification and characterization of biological structures and processes from image recordings are increasingly important in biomedical research. In many cases of image analysis, humans can perform a better job than the computer. However, human resources are expensive and can have severe limitations when it comes to 3-D or spatio-temporal data acquisitions. Moreover, methods based on visual inspection are subject to inter- and intra-observer variability and time consumption of manual methods can be prohibitive in many cases. In accordance with the instant invention an automated method is provided for detection of recently discovered cell to cell communication channels that can be imaged with modern live-cell 3-D fluorescence microscopy techniques.

Mammalian cells interact with one another in a variety of ways, for example, by secreting and binding diffusible messengers like hormones and growth factors, or, between attached cells, via gap junctions. These fragile, actin-rich structures were shown to transport organelles of endocytic origin from one cell to another in an uni-directional fashion. The tubules allowed the passage of vesicles of endocytic origin but excluded other organelles like mitochondria and also did not appear to allow significant transfer of cytosolic proteins [Baluska F et al., Gerdes H H & Rustom A, Landes Bioscience 2005]. Provided that TNTs are present in tissue they may have numerous implications in cell processes including the intercellular spread of immunogenic material, of pathogens and of morphogens during developmental processes. Similar structures in plants, the plasmodesmata, are of great importance for movement of signaling molecules between plant cells, and viruses seem to benefit from these structures when moving from one cell to another. The invention therefore provides a method and system which allows a direct study and, most importantly, a quantification of TNTs, which have many important tasks in the human cell system.

The occurrence of TNTs inside a 3-D image stack can usually be spotted by a trained eye. However, using human resources when collecting quantitative information about TNTs in large collections of data recordings is extremely demanding and expensive. A single TNT may as well appear in several image planes, requiring 3-D analyses in searching the image stack for TNTs. After the recent discovery of TNTs, cell biologists are now very interested to obtain more information about the formation and disappearance of TNTs, and whether they need special circumstances to appear or to disappear. When the basic functions of TNTs are known, we can monitor their role in pathogenesis of various diseases, such as in cell to cell communication during spread of cancer or viruses like HIV, or in immunological processes. If there were pharmaceuticals available for altering the formation or disappearance of TNTs, we could use these actively to induce biological responses, assessed by imaging techniques. Automated or semi-automated procedures for finding and characterizing TNTs in image recordings will thus be an important tool for facilitating TNT research.

Our approach for finding TNTs in microscopic images is based on binary classification of the image into cells and background. Once this has been established, we can use the property that TNTs are crossing from one cell to another. Detection and classification of cells in microscopic images is a large area of research, with a relative long history within biomedical imaging (e.g. Lynn M. et al., Elsevier, Science direct 2004, 16, 500; Wu K et al, IEEE Transactions on Biomedical Engineering 1995, 42:1-12; Nattkemper T W et al., Comput Biol Med. 2003, 33:31; Bengtsson E. et al., Pattern Recognition and Image Analysis 2004, 14:157-167). In some cases there are commercially available software packages for cell characterization and cell counting for clinical and research use (e.g. A. E. Carpenter and T. Ray Jones, “The cellprofiler, cell image analysis software project.” [Online]. Available: www.cellprofiler.org). However, it is important to keep in mind that these cell detection packages are very specialized, depending on specimen preparation, sectioning and staining, as well as imaging method, spatial resolution and what kind of cells and artifacts we are dealing with.

Wählby et al. in Analytical Cellular Pathology 2002, 24:101-111 obtained between 89% and 97% correct classification by using a watershed segmentation method with double thresholds for detecting CHO-cells in fluorescent microscopy images. They faced over-segmentation by merging small objects with their neighbouring objects, using the integrated pixel intensity of the objects to decide which objects to merge. The small objects were then merged with the neighbour having the highest summed intensity of touching borders. By calculating a Mahalanobis distance between feature vectors associated to the objects, they obtained a quality measure for the classification into cells, background and artefacts. For splitting of under-segmented objects they used the convex hull for locating concavities, assuming that cells have concave like shapes.

Yang & Jiang (Journal of Biomedical Informatics 2001, 34:67-73) proposed a method for segmentation using kernel-based dynamic clustering and an ellipsoidal cell model. They computed the gradient image to obtain points that likely belong to cell borders. A Gaussian based kernel was formulated for each clustering of regions, and each image point was devoted a probability to belong to a specific cluster or not. A genetic algorithm based on these probabilities was used to match regions from the gradient image to the ellipsoidal cell model. This model benefits from the fact that cells often have ellipsoidal shape, but that is not always the case. Further, occlusions are not necessarily well handled.

Mouroutis et al. [Bioimaging 1998, 6(2):79-91] proposed a method of finding possible locations of cell nuclei using a compact Hough transform (CHT). Their CHT assumes that the cells are convexly shaped, so that all boundary points of a cell lie within a maximal and a minimal distance from the nuclear centroid. Following the convex assumption, they assume that the nuclei will lie within one of the semi planes defined by the tangent of the boundary. A likelihood maximization was used in combination with the CHT to find the possible nuclear boundaries. They report good results for light microscope images using stained tissue sections. They claim encouraging results even for cases where the cells are dividing. However, no percentage for misclassification was presented.

Gamido and de la Blanco [Pattern Recognition 2000, 33:821] used deformable templates to identify cells under conditions with substantial noise. They applied a generalized Hough transform (GHT) with a relatively large region of uncertainty which was used to roughly detect round-like shapes. These elliptic structures were later used as input for the Grenader deformable template model to fit the cell borders more accurately.

TNT detection itself requires a fair amount of different approaches than those used for cell detection. Automated TNT detection has not been previously reported, and relevant detection problems with similar characteristics will therefore be discussed below. These problems deal with detection of straight line segments, partly using edge-detectors and Hough transformations. Nath & Depona [MATLAB 2004] applied Canny's edge detector to find edges of a DNA-protein, followed by an active contour model, a snake, for identification of the exact and connected curve surrounding the protein. However, the snake model could only detect one DNA-protein, even in the presence of many, and leaving it to the user to seed the snake initially. Niemisto et al. [IEEE Transactions on Medical Imaging 2005, 24(4):549-553] used image analysis methods to quantify angiogenesis which was influenced by stimulatory and inhibitory agents. Their method gave length and number of junctions of the tubule complexes, applying thresholding and thinning to detect the thin blood vessels. From quite another field, automated detection of bridges in high-resolution satellite images is a strikingly similar problem to our task of TNT detection. Lomenie et al. [Proc of the 2003 International Geosciences and Remote Sensing Symposium IGARSS 2003] reported a low rate of false positive (around 5%) but also a low success rate (around 40%) for their algorithm. They explored both textural and geometric approaches. The textural approach was used to classify each pixel into type of terrain using an neural network, and thereafter they applied selection rules to the image. Their geometric approach was based on edge filtering and search for parallel neighbor-segments as candidates for bridges. For the same problem, Jeong and Takagi [Proceedings of the 23^{rd} Asian Conference on Remote Sensing, Kathmoandu 2002; (172)] used a Prewitt filter and Hough transformation to detect the bridge constructions that appear as straight lines.

Several ideas from the previous work described above like watershed segmentation, Hough transformation and edge detectors, have been applied for the task of TNT detection and quantification. However, finding so extreme thin structures as TNTs automatically, is such a great challenge that in addition to the cell borders, the cell interior had to be labeled by a fluorescent marker. This cell marker created a second image channel, marking the cells as light regions and background as dark regions. The cell marker itself provides not sufficient information to distinguish each cell from other cells, but it can distinguish cells from background. The processing steps presented in this paper are developed in order to enable identification of which pair of cells each TNT is connecting. The chain of processing steps we have designed, incorporates generic methods from digital filtering (incl: deblurring with Richardson-Lucy deconvolution), edge detection (Canny's edge detector) and mathematical morphology (incl. watershed segmentation). All algorithms at different steps are implemented for 3D images, either using entirely 3D based operations, or assisted by specialized projections, assimilating 3D information into 2D images.

For the present task of TNT detection and quantification, we have tried to employ several ideas from previous work described above, but finding such fragile and thin structures as TNTs automatically, is such a great challenge that we decided to go for a biological cell marker additionally. This cell tracker will mark the cells in a separate channel as light regions whilst background is darker. However, the cell tracker can not provide us with sufficient information when distinguishing cells from each other. The processing steps presented in this paper are developed in order to make it possible to detect TNTs in a image. Additionally, the program identifies exclusively for each image which cells the TNTs are connecting. Therefore we decided to combine the biological cell tracker with several image processing techniques described above, in order to characterize both TNTs and cells. We have designed a chain of processing steps incorporating generic methods from digital filtering (e.g. deblurring with Richardson-Lucy deconvolution), edge detection (e.g. Canny's edge detector), ridge enhancement and mathematical morphology (e.g. watershed segmentation). All algorithms at the different steps are implemented for 3-D processing. Our automated method was compared to manual segmentation (taken as “ground truth”) and applied to a total of 40 3-D datasets. Using a hold-out method, separating data used for model selection (training and parameter estimation) from data used for performance estimation, we obtained, on average, a success rate of 75% and greater than 90% with a ridge enhancing curvature filter. The ridge enhancement can also be applied to the image and then be followed by an adaptive thresholding. For research use, in this early stage of TNT history, we find this acceptable, taken into account the cost, time consumption and observer-variability of using manual TNT counting.

Further advantages, objects and features of the invention are provided in the examples and the accompanying Figures

### BRIEF DESCRIPTION OF THE DRAWINGS

FIG. 1 shows representative microscopic images taken from the same plane of mono-layer PC12 cells used for TNT detection. (a), (c), (e), (g) show TNTs (marked by arrows) spanning between cells and cell borders and (b), (d), (e, (h) the cytoplasmic area of these PC12 cells—white bar in (a) corresponds to 5 micrometers;

FIG. 2 shows a schematic flow scheme of the method for automated detection of TNTs;

FIG. 3 shows a segmentation of cellular regions of FIG. 1(a) into a binary mask. The cell marker image (a) has been segmented into extracellular (black) and intracellular (white regions);

FIG. 4 shows that edge detection leads to the identification of cell borders and TNT candidates. Canny's edge detector was applied to the image in (a), resulting in a binary image (b) showing all edge components—the edge component used for further demonstrations is labeled with an arrow;

FIG. 5 shows a maximum projection of a TNT candidate from the edge image. The original image (a) shows a TNT. The corresponding maximum projection of its edge structure is seen in (b), which originates from the edge structure indicated by an arrow in FIG. 4(b). The maximum projection was later used for initializing a watershed segmentation.

FIG. 6 depicts the minima seed regions for watershed segmentation. The sum image in (a) has a TNT candidate between the corresponding minima seed regions in (b). These seed regions were used for initializing a watershed segmentation to detect the ridge of the TNT candidate.

FIG. 7 shows the ridge of a TNT candidate and the cell borders have been found from watershed segmentation of FIG. 6(a) using the initialization regions in FIG. 6(b).

FIG. 8 shows the initialization regions for watershed segmentation of cells. The image (a) is assigned a minima marker image (b) that initializes the watershed segmentation of cells.

FIG. 9 shows watershed segmentation of cells. The image shows the borders between the regions that appear from watershed segmentation of FIG. 8(a). Two regions marked with arrows are incorrectly assigned as individual regions due to over-segmentation.

FIG. 10 shows the classification into cells, TNT candidates and cell borders. White regions are cells, the grey lines important edges, i.e. cell borders, TNTs and artefacts, and the black regions background.

FIG. 11 shows the result of a checking whether the TNT candidate is a high-intensity edge or a flat region. A narrow, bilateral neighborhood following the TNT candidate defines a close neighborhood around the TNT candidate. The mean image intensity corresponding to the neighborhood pixels was compared to the mean image intensity on the TNT candidate itself.

FIG. 12 shows the final detection of TNTs. All TNTs labeled by arrows in (a) have been automatically detected in (b).

FIG. 13 shows a microscopic image of sharp edged filopodia-like cell structures (marked by arrows). Most false-negative and false-positive automated TNT detections are due to high intensity image structures resembling TNTs. The case of cells close to each other is particularly challenging.

FIG. 14 shows a graphical representation of the distribution of the 3D length of automatically detected TNTs. Small TNTs between 1 μm and 4 μm connecting close cells are dominating.

FIG. 15 shows a flowscheme of segmentation wherein the input image is filtered further using a ridge enhancing curvature filter. Then, the markers for watershed segmentation are created from flood filling, and the watershed segmentation is applied. Insignificant watershed borders are removed, and finally the segmented regions are classified into cells and background.

FIG. 16 shows an image of surface stained PC12 cells. The plasma membranes are expressed as ridges.

FIG. 17 shows a schematic representation of topological variations. The plasma membranes are typically characterized by ridges (a), and not by valleys (b), peaks (c) or holes (d).

FIG. 18 shows a representation wherein the image (a) has been transformed into (b) through the ridge enhancement. (c) and (d) display the line profile of the labeled line of the image and the ridge enhanced image, respectively. This clearly demonstrates how the ridge enhancement raises the contrast of the ridges compared to other structures in the image.

FIG. 19 shows a cell image after flood filling. The holes of FIG. 18 have been filled, creating constant valued regions.

FIG. 20 shows the creation of a minima marker image. The piecewise constant image in FIG. 19 is transformed into a binary marker image which is used for marker controlled watershed segmentation.

FIG. 21 shows a watershed segmentation of cells. A marker controlled watershed segmentation is performed on the ridge enhanced image in FIG. 20, and the watershed lines achieved are shown in (a). The piecewise constant watershed image (b) depicts each connected region labeled by a unique integer.

FIG. 22 shows a classification of cells. The watershed regions in FIG. 21(b) are classified as cells (white) and background (black). One of the watershed lines are wrongly removed by the significance test, thus embedding an error in the classification, shown by an arrow. The displayed region should correctly have been divided into two regions, one cell region and one background region.

FIG. 23 shows a bad co-localization of borders around segmented regions. The left image is segmented, giving the right image. The number of regions equals three for both, but the borders around the segmented objects are misplaced. This demonstrates that an appropriate measure for correctness of segmentation must comprise both the number of segmented regions and the co-localization of their area.

FIG. 24 shows a graphic representation of the measuring correctness of regions overlap. Solid lines surround the reference regions, and the dotted lines outline the automatically segmented regions. (c) is the perceptually best segmentation, in accordance with the highest similarity measure of 0.91 in Table 1

FIG. 25 shows the image in FIG. 24(a) has been manually (grey lines) and automatically (white regions) segmented. The similarity measures reflect different quality of the segmentation. The segmentation for (a) is poor (SM=0.007), for (b) fair (SM=0.663), for (c) good (SM=0.861) and for (d) fair (SM=0.678).

FIG. 26 shows a selection of four representative images used for cell detection. Each image is one 2D plane taken from the middle of its 3D image stack. The bar in (a) corresponds to 10 μm.

FIG. 27 shows a selection of two representative spinning disc images showing WGA stained NRK cells used for cell detection. Each image is one 2D plane taken from its 3D image stack. The bar in (a) corresponds to 20 μm (pixel size: 0.2048 μm×0.2048 μm).

FIG. 28 shows photographs of two representative confocal images taken with the Leica SP5 showing WGA stained NRK cells used for cell detection. Each image is one 2D plane taken from its 3D image stack. The bar in (a) corresponds to 20 μm (pixel size: 0.283 μm×0.283 μm).

FIG. 29 shows two representative images from f-EGFP stained PC 12 cells used for cell detection. Each image is one 2D plane taken from its 3D image stack. Note the large drop-out of membrane fragments in the left image. The bar in (a) corresponds to 20 μm (pixel size: 0.1340 μm×0.1340 μm).

FIG. 30 the input image (A) for ridge enhancement, the ridge-enhanced image (B) and the binary image (C) created from adaptive thresholding. The ridge enhancement is applied to the image and then followed by adaptive thresholding.

### DETAILED DESCRIPTION OF THE INVENTION

Cultured PC12 cells are 3D objects forming a network of TNTs. Due to the distribution of plated cells, the TNTs are mainly propagating in the xy imaging plane. However, they are sometimes inclined, requiring a 3-D tool for TNT detection. Our algorithm takes advantage of these properties of the TNTs, by applying projections from 3D to 2D. Provided that TNTs exist in tissue, which is left to be shown, their straight line appearance could change into bended structures due to the dense extracellular matrix. Further, one could expect TNTs to propagate equally in all spatial directions. Thus, for a tissue sample, a rotationally invariant approach would be necessary to detect TNTs.

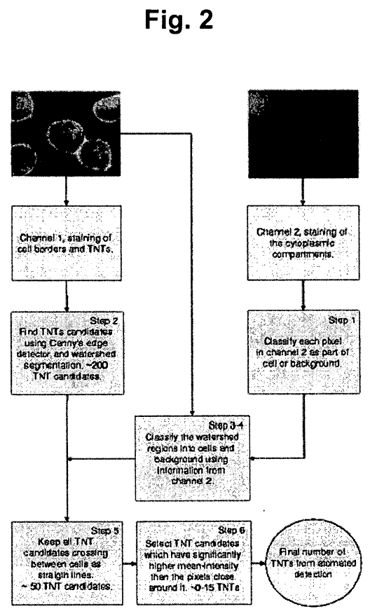

We approached the problem of finding TNTs by searching the image for all important edges occurring on background regions. Thereafter we employed several properties of TNTs to locate them and for the removal of false candidates appearing from edge detection. TNTs are tube-like structures from one cell to another crossing background, which is the property that can be used for clear identification. The robustness of the algorithm depends critically on its ability to classify the segmented regions into cells and background with high accuracy, and we accomplished this using a biological cell tracker. For plated PC12 cells, we searched the image for all significant edges occurring on background regions since TNTs are intercellular structures. As a first preprocessing step, deblurring using Richardson-Lucy (R-L) deconvolution [Carasso A S, SIAM J Numer Anal 1999; 36(6): 1659-1689 (electronic)] was performed, assuming the focal plane images are Gaussian-like blurred. This iterative image restoration algorithm is based on maximizing the likelihood of the resulting image being an instance of the original input image under Poisson statistics. In all experiments, the R-L algorithm was supplied with a Gaussian point spread function (PSF) of size 5×5 pixels and standard deviation 5. A general outline of the control flow of our algorithm, omitting the initial image restoration step (R-L deconvolution), is given below (see flow scheme of algorithm in FIG. 2)

In essence, the Canny's edge detection method is used to discover important edges in the first image channel. All edges found inside these regions belong to cells and can be ruled out as TNTs. The remaining ones are used as input for a 2-D watershed segmentation of a depth projection to accurately find the crest of the edges. The cells are marked in the first image channel using flood filling. Thereupon the cell borders are detected using a 3D watershed segmentation. Correcting errors in the watershed image such that all edges are one pixel wide and form closed contours. The found edges and cells are combined into one single image displaying all cell borders and possible TNTs. Then, the structures are selected that are candidates for TNTs, namely on basis of the property that a TNT must cross background. This is followed up by reducing further the number of candidates for TNTs by keeping those crossing from exactly one cell to another, and discarding the others. In a further step, the number of candidates for TNTs is reduced by keeping those being straight lines and rejecting the rest. At this stage we had also ensured that the intensities of each candidate is significantly higher than the intensity of the pixels close to it. With regard to the flow scheme of this algorithm for automated detection of TNTs, please refer also to FIG. 2.

In essence, the cell tracker channel provided us with information on cell distribution and background. Thus, we obtained from this channel a minima image marking of the inside and the outside of cells. The maximum image for each connection produced by an edge detection was then projected upon this minima image after morphological closing to produce a final minima image as input for a watershed algorithm. As the TNTs are frequently crossing multiple planes, we used a sum image of the original image for watershed segmentation. Again, the 3-D information was projected onto a 2-D space so that the problems by TNTs crossing several planes were minimized as the TNTs were now visible in the 2-D projection over their entire length. Additionally, the sum image resulted in noise removal when ranging over a limited number of planes while keeping TNTs visible. The total sum image, however, can not be applied to all planes in the whole image stack since that would blur again the TNTs to invisibility All projections from 3-D to 2-D must therefore use the same range. A watershed segmentation was then applied to the projected sum image using the minima image as seeding points for the algorithm. The watershed segmentation was performed for each one connection at a time to avoid different connections binding to each other. If binding happens, some connections found by the edge detection were undesirably removed. Then, strong criteria were then applied onto the TNT candidates found by edge detection and subsequent watershed segmentation, so that each one connection was classified as a TNT or not.

For watershed segmentation of cells, the cell image is divided into meaningful regions separated by high-intensity edges. The watershed transformation groups image pixels around regional minima of the image and the boundaries of adjacent groupings are precisely located along the crest lines of the gradient image. Watershed is best suited for images with natural minima. However, direct application of the watershed transformation to a grayscale image ƒ often leads to over-segmentation due to noise and small irregularities. To limit the number of allowable regions, we incorporated a preprocessing step to control the flooding process for given ƒ. A marker image will have a set of internal markers consisting of connected components that are inside of the objects of interest, and assigned to a constant mean value of that region. The result then depends highly on the marker image. To obtain our ƒₘ, we filled all minima in ƒ that were not connected to the image border. These connected, constant-valued regions inside the objects of interest were denned by the zero gradient of the ƒₘ image. Using minimum marker images, we achieved a watershed transformation with an acceptable degree of over-segmentation, only including some undesired irregular edges that were not representing cell borders. Each connected region from this watershed segmentation is called a watershed region, which are then classified into cells and background.

TNTs crossing background is an important exclusion criteria for the TNT candidates processed from the edge detection. We therefore classified the connected watershed regions into either a cell or part of the image background. From the second image channel, the cell tracker channel, we obtained the data which parts of the image are cells and which not. The obtained grayscale image was then converted by several processing steps into a binary mask. After noise reduction and Canny's edge detection on the cell tracker channel, the closed contours surrounding high-intensity regions were filled and a binary cell image created wherein cells are white and background black. The cell tracker channel does not allow, however, an accurate tracing of cell borders but can mark borders adjacent to background. As we wished to know between which cells TNTs are crossing, we did a detailed classification of all watershed regions. Classification of the watershed regions is straightforward. Each region is placed on top of the binary cell image and the region is classified as a cell if it is covering more cell-classified pixels than background-classified pixels. False classification of watershed regions is rare. A further step is the localization of edges crossing background. We extracted all edges crossing background since we could expect to find TNTs there.

A morphological dilation of the cell regions gave the TNT candidates. TNTs appear as straight lines crossing background from one cell to another. We took advantage of this property by the setting that TNTs must extend between exactly two cells. Dilation of the TNT candidates resulted in some overlap with the surrounding cells in the cases where the candidates were nearby the cells. By counting the number of cells covered by these dilations, it can be determined whether the TNT candidate is crossing between exactly two cells or not. The dilation was performed iteratively up to a specified maximum threshold. Moreover, we calculated the maximum Eulerian distance between all points in each TNT candidate. Comparing that distance to the number of pixels in the skeletenized connection, we could, based on a threshold technique, decide whether the TNT candidate is more or less a straight line or not. In some cases several TNTs are originating from one spot into a fan-like shape, if this structure is interpreted as a single structure, the test may fail. We then checked whether all TNT candidates have higher grayscale values. A TNT is characterized by moderate grayscale values in a global sense, but locally their intensity values will be significantly higher right on the TNT than compared to the surroundings. A subtraction of the image intensities of two almost equal dilations of the TNT candidates defines a close neighborhood. The grey-scale intensities on each TNT candidate is compared to the intensities of its neighborhood. Insignificant differences imply removal of the TNT candidate as false positive TNT. In some cases, artificial candidates pass through all preceding tests, candidates that are practically too small to be a TNT, covering only a few pixels. These are removed using a simple threshold value for the largest distance between the points in the candidate, they are anyway too short to undergo a correct TNT evaluation.

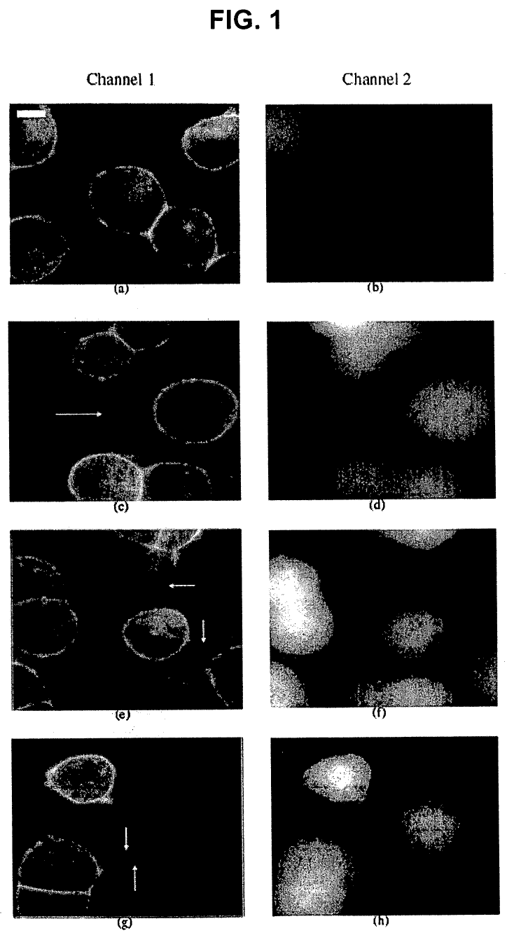

All algorithms and statistical evaluations in this paper were implemented in MATLAB 7.0.1 and executed on a 64-bits AMD processor 2.2 GHz running Linux. An average process took approximately 20 minutes for a 3D stack. MATLAB was chosen for the implementation due to its broad library of built-in image processing functions. The code in our algorithm has been extensively vectorized to obtain computational speed, probably at the same order as compiled code. In the following, details from each processing step are described. The results from each step as they to the data of FIGS. 1(a-b) are illustrated.

### EXAMPLES

### A. Preparation of the Microscopic Images

All image analyses were applied to mono-layers of cells from the living rat neuroendocrine cell line PC12 (rat pheochromocytoma cells, clone 251, gift of R. Heumann). This cell line was first generated in 1976 by Greene and Tischler [PNAS USA 1976; 73:2424-2428] from a transplantable rat adrenal pheochromocytoma. It is a single cell clonal line which grows monolayer forming small clusters. The PC12 cells also represent a common convenient model system for the study of secretory, neuron-like cells in cell culture. For comparative studies, NRK cells (normal rat kidney, Mrs. M. Freshney, Glasgow, UK) were used.

PC12 and NRK cells were cultured in DMEM supplemented with 10% fetal calf serum and 5% horse serum. For high-resolution fluorescence microscopy and light microscope analysis, PC 12 cells were plated in LabTek™ chambered swell cover glasses (Nalge Nunc Int., Wiesbaden, Germany). Two hours after plating, the cells were stained with two dyes. For the experiments in which the effect of thymidine on cellular size and morphology was investigated, PC12 cells were plated on LabTek™ chambered swell cover glasses. 24 hours after plating, fresh growth medium containing 4 mM thymidine (Sigma) was added to the cells. In the control condition, fresh growth medium without thymidine was used. 24 hours afterwards cells were washed once with prewarmed fresh growth medium and grown further in growth medium without thymidine. 24 hours after exchanging the medium, cellular surfaces were revealed by staining the cell monolayers with dye-conjugated wheat germ agglutinin (WGA) and by performing 3D fluorescence microscopy (see below). To specifically display cell borders, cells were stained with wheat germ agglutinin (WGA) conjugated to either AlexaFluor™488 or AlexaFluor™594 (Invitrogen). WGA-AlexaFluor™594 is a lectin which binds glycogenfugates like N-acetylglucosamine and therefore stains biological membranes efficiently. CellTracker™ (CellTracker™, Molecular Probes Inc., Eugene, Oreg., USA) passes freely through cell membranes, but once inside a cell, it is transformed into cell-impermeant reaction products and is retained in living cells through several generations. For the cytoplasm staining, CellTracker™ Blue Solution (20 μM final concentration) was added directly to the culture medium of an approximately 80% confluent 15 cm culture dish. Then the cells were transferred to LabTek™ chamber 4-well cover glasses in an appropriate dilution and incubated for three hours at 37° C. and 10% CO₂. For the plasma membrane and TNT staining, WGA conjugates (1 mg/ml) were added directly to the culture medium ( 1/300) before microscopy.

High resolution, bright-field fluorescence microscopy was performed with an Olympus IX70 microscope (Olympus Optical Co. Europa GmbH, Hamburg) or a Zeiss Axiovert 200M (Bergman A S, Lilleström, Norway) both equipped with 100× oil-immersion objectives, monochromator-based illumination systems (T.I.L.L. Photonics GmbH, Martinsried, Germany), tripleband filtersets DAPI/FITC/TRITC F61-020 (AHF Analysetechnik AG, Tübingen, Germany) and piezo z-steppers (Physik Instrumente GmbH & Co., Karlsruhe, Germany). The imaging system was also equipped with a 37±C heating control device and a 5% CO₂ supply (Live Imaging Services, Olten, Switzerland). Confocal microscopy was performed either with a spinning-disc imaging setup (Perkin Elmer UltraView RS Live Cell Imager) installed on a Zeiss Axiovert 200 microscope or with a Leica TCS SP5 confocal microscope (Tamro, Oslo, Norway) using the resonant scanner for fast image acquisition. Image recordings were performed at excitation wavelengths of 488 or 555 nm for the AlexaFluor™488- or AlexaFluor™594-conjugates of WGA, respectively. With both the wide-field and confocal imaging setups, WGA-stained cells were analyzed in 3D by acquiring single focal planes 300 to 400 nm apart from each other in the z-direction spanning the whole cellular volume. Images acquired with the wide-field setups were first converted to grayscale images using the integrated autoscale macro in the TILLvisION software (T.I.L.L. Photonics GmbH, Martinsried, Germany), saved as 16 bits TIFF images, 134 nm×134 nm or 129 nm×129 nm pixel size and 520×688 image dimensions. Confocal imaging at the spinning disc resulted in 16 bits TIFF images of 512×672, each pixel having an extension of 201×201 μm. Single images from 3D stacks acquired with the Leica SP5 setup were exported as 8-bit grayscale tif images with a resolution of 4512×512 and 283.22 nm×283.22 nm pixel dimensions. Dual channel image recordings were performed, the first channel at a wavelength of 555 nm recording the WGA AlexaFluor™, the second channel at a wavelength of 400 nm recording the CellTracker™ Blue signal. For each channel, 40 planes were acquired, processed by using the deconvolution extension of TILLvisION and resulting in stacks of grey-scale unsigned integer 16 bits images with dimensions 520×688×40. Each pixel had an extension of 134 nm×134 nm, summing up a total image area of 69.68 μm×92.19 μm, and the separation between the focal planes was 300 nm.

### B. Input Data and Processing Steps in The TNT Segmentation Procedure

To illustrate the type of data, a selection of four representative dual channel images belonging to separate 3-D image stacks are shown in FIG. 1(a-h). Notice the presence of noise, uneven illumination and intracellular grains of similar intensity as cell borders in the left column of these images. Clearly visible TNTs are marked with arrows. These images represent the first and second image channel from a given focal plane, zoomed larger to display the fine details. For practical reasons merely one single plane from each image stack is shown. The left column shows the first image channel, and the right column shows the corresponding second image channel displaying cells as bright regions. The second image channels was used to separate cells from background at high contrast. It allows to eliminate TNT candidates detected in cellular areas.

As apparent from the images of FIG. 1 TNTs are very thin, elongated structures, appearing as almost straight lines connecting one cell to another. Typically, the width of TNTs seen in fluorescent images is comparable to one third of the thickness of imaged cell walls. The TNTs have notably darker grey levels than the cell walls, and their grey-level and noise characteristics vary little along their extension in 3-D. They are surrounded by darker intercellular regions except at their endpoints where there is a seamless connection with the plasma membrane. The image recordings, however, are hampered by moderate noise and blurring of fine details, and in certain cases TNTs are located very close to each other, as in Figure I(g). In rare cases it is hard to decide, even by a trained eye, whether a structure is a TNT or not. As a consequence, automated TNT detection is a challenging image analysis task. Cultured PC 12 cells are 3D objects forming a network of TNTs. Due to the distribution of plated cells, the TNTs are mainly propagating in the xy imaging plane. However, they are sometimes inclined, requiring a 3D tool for TNT detection. Our algorithm takes advantage of these properties of the TNTs, by applying projections from 3D to 2D. Provided that TNTs exist in tissue, which is left to be shown, their straight line appearance could change into bended structures due to the dense extracellular matrix. Further, one could expect TNTs to propagate equally in all spatial directions. Thus, for a tissue sample, a rotationally invariant approach would be necessary to detect TNTs.

For plated PC 12 cells, we have chosen to approach the detection problem by searching the image for all significant edges occurring on background regions, since TNTs are intercellular structures. As a first preprocessing step, deblurring using Richardson-Lucy (R-L) deconvolution [Carasso A S. in SIAM J Numer Anal 1999; 36(6): 1659-1689 (electronic).] was performed, assuming the focal plane images are Gaussian-like blurred. In all experiments, the R-L algorithm was supplied with a Gaussian point spread function (PSF) of size 5×5 pixels and standard deviation 5. A general outline of the control flow of our algorithm, omitting the initial image restoration step (R-L deconvolution), is given in FIG. 2. In the following, details from each processing step are described. The results from each step as they apply to the data of FIG. 1(a-b) are illustrated.

### C. Description of Each Processing Step

### C1. Classification of Cells and Background

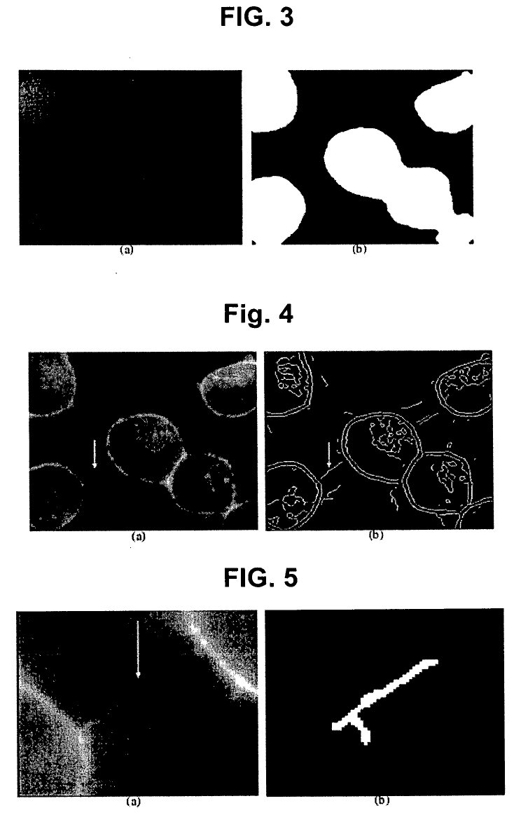

The cell marker channel was used for binary classification of each pixel into cell or background. As seen in FIG. 3(a), the cell soma appears as high intensity regions in the cell marker channel. Applying a simple threshold for segmentation of cells is unsuitable due to noise and uneven illumination. The boundaries of the cells are better characterized using an edge detector. Canny's edge detector was therefore used to mark the border between cells and background, and the closed regions were filled using morphological filling. By these means, a partition into “intracellular” and “extracellular” regions was obtained, displaying cells as white and background as black. The result of this processing step, applied to FIG. 3(a), is shown in FIG. 3(b).

### C2. Detection and Identification of TNTS

TNTs are structures occurring at a certain level above the substrate and they are usually not found in the uppermost planes of the 3D images from PC12 cells. Thus, the algorithm has been applied exclusively to the central 30 planes of the stacks, discarding the upper five and lower five planes in each stack to restrict computational time and reduce the number of false-positive TNT candidates. In other words, all calculations were based on 30 planes of the image stack, ranging from plane 5 to plane 35, although the image stack had 40 planes. This decision is justified since TNTs are both structures occurring at a certain level above the substrate, as well as empirically not found in the uppermost levels of the stacks of PC12 cells. At each processing step, for the sake of displaying, we only draw the most interesting plane. TNTs are structures with moderate grey-scale values compared to cell borders. Consequently, searching and screening for TNTs using entirely intensity based segmentation algorithms will therefore fail. However, they are thin and elongated with a relatively high gradient normal to their pointing direction, and therefore Canny's edge detector was applied to channel 1, thus highlighting important edges. This process, exemplified for FIG. 4(a), is shown in FIG. 4(b).

Removal of the smallest components of the edge image made by the edge detector still left numerous false TNT suggestions for structures arising from natural edges in the original image. The smallest edge components were removed by thresholding since they were below the size limit for a reasonable evaluation. As a first step in the edge pruning, all edges inside the cells were removed, and the connected components outside the cells were labeled individually using first order neighborhood. To retain 3D information for each component into a 2D image, the maximum intensity projection (MIP) was applied. In brief, assume that ƒ is the 3D-image of the first channel. The MIP maps the image planes between ƒₘ and ƒₙ into a 2D-image which takes the maximum intensity values along the z-direction. The maximum projection was calculated for each connected component in the edge image, the component ranging from plane m to n. The MIP was thus restricted to a limited number of planes. The maximum projection ρₘₐₓ(ƒ, r₁, r₂) for each one is calculated and projected onto a 2-D plane. This projection ρₘₐₓ (ƒ, r₁, r₂) is therefore a maximum projection of the 3-D image ƒ onto a 2-D plane, ρₘₐₓ(ƒ, r₁, r₂): [symbol] ³→ [symbol] ² where the 3-D image used in the projection is ranging from plane r₁ to r₂. The range (r₂-r₁) is normally less than the total image dimension of the whole image stack, typically ranging over a few planes. In the process of calculating the maximum image for each connected component, we used only the planes over which this connection is continuously connected. Thus we avoided artifacts from other connections that are not connected to this specific one. Further, the original image is reduced in xy direction for these calculations, if not, the watershed segmentation may in some cases fail in locating the TNT candidate. FIG. 5(b) depicts the maximum projection of the component indicated by the arrow in FIG. 4(b). The image region corresponding to FIG. 5(b) is shown in FIG. 5(a).

The cell regions (cf. FIG. 3(b)) and the eroded background regions were added into one single image. This created a binary image marking the inside and outside of the cells, omitting the cell borders. The projected structure of FIG. 5(b) was subtracted from this binary image, and a morphological opening was performed to open up a pathway from one cell to another in the cases where it was possible. This created a final marker image, used as initialization to a watershed segmentation (Gonzalez R C et al., in Digital Image Processing. Addison-Wesley Publishing Company; 1992; Soille P. in Morphological Image Analysis: Principles and Applications. Berlin: Springer-Verlag; 1999; Vincent L et al, IEEE Transactions on Pattern Analysis and Machine Intelligence 1991; 13(6):583-598) for each connected component in the edge image. The watershed segmentation was employed to locate the crest lines of the high intensity edges. The minima marker image corresponding to the structure in FIG. 5(b) is shown in FIG. 6(b) where the minima initialization regions are labeled white.

Furthermore, only image regions close to the structure of interest were used in further calculations to save computational time and increase accuracy of the watershed algorithm. The watershed segmentation required boundaries of the minima marker regions that were sufficiently close to the edge structure of interest, if that was not the case, the watershed segmentation would often detect another crest of minor interest, still containing strong edge information.

TNTs are frequently crossing several planes. Therefore the sum image from plane m to n was used as input for watershed segmentation. Let ƒ be the 3D-image of the first channel. For given m≦n, let ƒᵢ, i=m, . . . , n be plane i from the image stack. The sum projection ρₛᵤₘ (ƒ; m,n) is defined as

$${p}_{sum} ( f : m , n ) = {\sum }_{m \leq i \leq n} {f}_{i} . \qquad ( 1 )$$

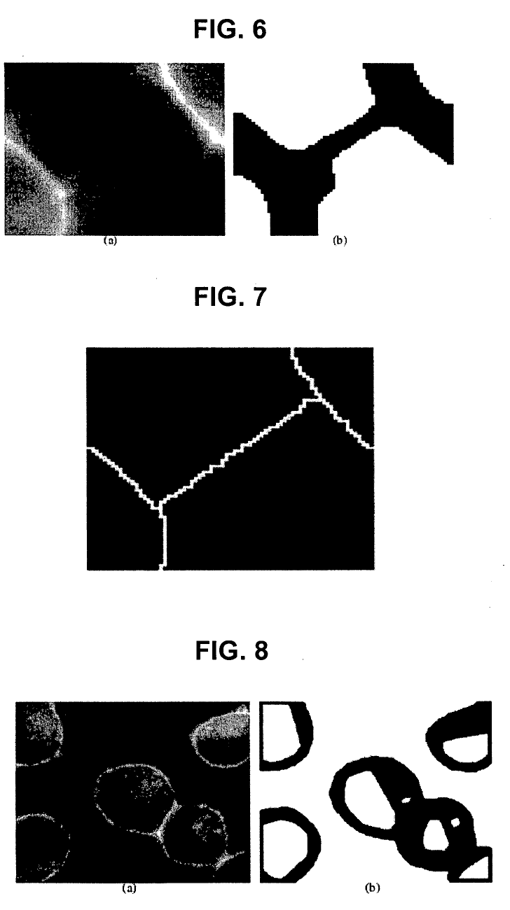

This projection maps the image planes between ƒₘ and ƒₙ into a 2D-image which adds the intensity values along the z-direction. Consequently, the problems of TNTs frequently crossing several planes was minimized as the TNTs now were visible in their whole length inside the 2D projection. Additionally, when adding multiple image planes close to each other, a stochastic noise suppression was obtained since the noise is assumed close to Gaussian and independent (when the effect of deconvolution is ignored). Summing all image planes in the 3D stack would blur the 2D projection too much, and at the same time blurring the TNTs. The projections from 3D onto 2D were therefore limited to the same range as the current structure found by the edge detection, thus enhancing the edge feature that was investigated. A normalization of (1) is possible, but not necessary, since a scaling factor will not influence the forthcoming watershed segmentation. A watershed segmentation was applied to the projected sum image in FIG. 6(a) using the minima image in FIG. 6(b) as initialization for the algorithm. The watersheds created, are depicted in FIG. 7, labeling the ridge of the structure of interest.

The watershed segmentation was repeated for each and every edge structure in the edge image. It was not possible to perform the watershed segmentation for all connections simultaneously, since information would then get lost from the morphological opening in the case of close structures.

### C3. Watershed Segmentation of Each Cell

In section C1, the image regions covered by cells and background were acquired from the second image channel. However, this segmentation provides insufficient information about cell-to-cell borders of associated cells, only outlining the cell-to-background borders (cf. FIG. 3(a)). Therefore, to obtain an algorithm being able to determine between which pair of cells a TNT is crossing, a specific cell-by-cell segmentation was additionally required. To partition the first image channel (FIG. 8(a)) into meaningful regions that are separated by high intensity cell walls, a watershed transformation was used. The method is well described in literature (Vincent L et al, IEEE Transactions on Pattern Analysis and Machine Intelligence 1991; 13(6):583-598; Lin Umesh G A et al., Cytometry, Part A 2003; 56A(1):23-26; Adiga PSU, Microscopy Research and Technique 2003; 54(4):260-270), and the largest disagreements arise from the problem of creating suitable minima to initialize the watershed algorithm. Direct application of the watershed transform to a gray-scale image ƒ often leads to severe over-segmentation due to noise and image irregularities. To obtain the marker image, all minima in ƒ not connected to the image border were filled. This was performed by filling the holes in ƒ ([23, pp. 173-174]) using morphological reconstruction by erosion [Vincent L., IEEE Transactions on Image Processing 1993; 2:176-201] as implemented in MATLAB's Image Processing Toolbox. One example of such binarized marker image is shown in FIG. 8(b), created for image fin 8(a).

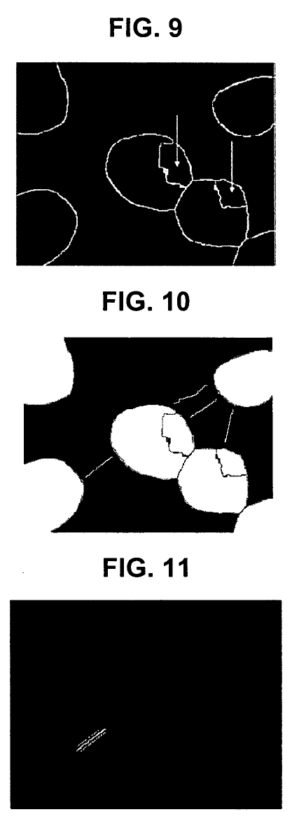

The markers representing the background were verified using the complement of the cellular areas computed in section C1, representing high-accuracy markers for the background. When using minimum marker images, the watershed transformation resulted in a certain degree of over-segmentation. Each connected region from the watershed segmentation is named a watershed region. FIG. 9 shows the borders between the watershed regions from FIG. 8(a). Notably, two small regions represent over-segmentation (FIG. 9, arrows).

### C4. Classification of Cells and Background

In order to decide whether a particular TNT connected two cells, the watershed regions were classified as cells or background using the information of channel 2. Each region was placed on top of the binary cell image (cf. FIG. 3(b)) from step C1, and regions were classified as cells if they covered more cell—than background-pixels. FIG. 10 depicts the classified regions of the watershed image in FIG. 9.

### C5. Straight Line Criteria of TNTs, Crossing Between Cells

TNTs are structures crossing on background from one cell to another, and it was checked whether this was true for each TNT candidate. The structure was dilated iteratively up to a predefined threshold, and the number of cells covered by the dilation were then counted, giving the number of cells close to the TNT candidate. Moreover, the Hough transformation for each TNT candidate was calculated. By comparing the minimum Hough transformation to a predefined threshold, it was decided whether the TNT candidate was approximately a straight line or not. If the connection was not a straight line, it was rejected as a TNT.

### C6. High Intensity Criteria of TNT Candidates

A TNT is characterized by moderate gray-scale values in a global sense, but locally their intensity values will be higher compared to their surroundings. A subtraction of the image intensities on two almost equal dilations of the TNT candidate, defined a narrow neighborhood on each side of the connection. This is illustrated in FIG. 11 where the TNT candidate is surrounded by the two lines following it. The gray-scale intensities on each TNT candidate was compared to the intensities of its bilateral, narrow neighborhood. Insignificant differences implied removal of the TNT candidate as a false-positive TNT.

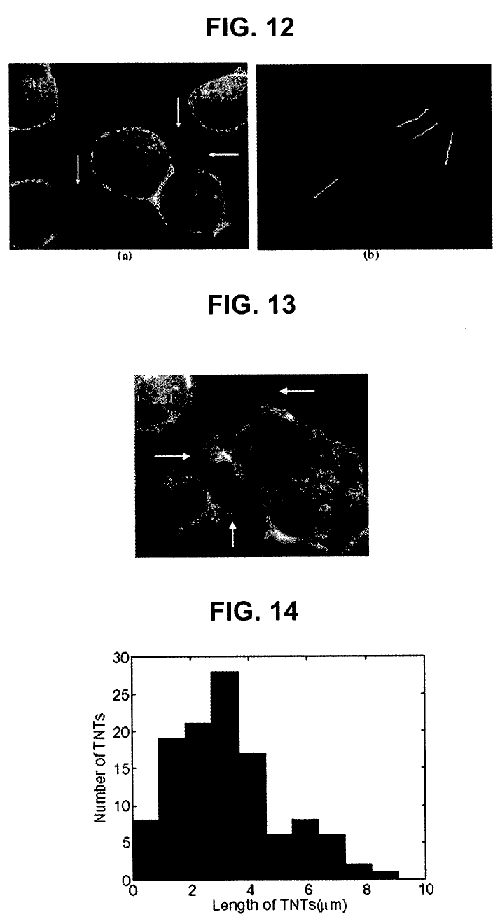

In some cases, artificial candidates passed through all preceding tests, candidates that are practically too small to be a TNT, covering only a few pixels. These were removed using a simple threshold value for the largest distance between the points in the candidate, they were anyway too short to undergo a correct TNT evaluation. The assumed real TNTs found at this stage, are shown in FIG. 12(b).

### C7. Method for Performance Evaluation

To test the robustness of our algorithm and avoid over-fitting to specific image data, it has been tested on a separate data set not used for design and tuning of the numerical routines. A “true” identification of TNTs, obtained by manual labeling and counts have been performed by two different observers. One of them, (S.G.), an expert on TNT biology, was not involved in the algorithmic development or the computer vision experiments. The other person (E.H) has been responsible for the development of the automated method. In the cases of doubt, the manual counting rules were such that the TNT candidate in question was discarded. For a connection to be regarded as a true TNT, it must have been rated as TNT by both human observers. A false-positive TNT detection is the situation where an image feature is found to be a TNT by the program, but not rated as a TNT by the observers, or at most by one of the observers. A false-negative TNT detection occurs when both observers decide the structure to be a TNT, but the program misses. Note that this method for performance evaluation imposes a very strong criterion of success for the algorithm since it is calculated from the number of agreements of both the human raters. Thus, the success rate of the automated method will be a very conservative estimate.

### C8. Experimental Results

The performance of the automated detection of TNTs has been compared to manual TNT identification. Using the hold-out method for performance evaluation and the counting rules described below, the automated detection was capable of locating 67% of the TNTs counted manually by two observers. The quality of the detection was evaluated by comparison with a manual counting of the TNTs in the original images. When the program failed to find a TNT, it was counted as a false negative. When the program found a TNT that did not exist in the manual counting, it was registered as a false positive. A structure was manually registered as a TNT only in the cases where there is no doubt. The manual counting was done by persons not involved in the development of the program. False-positive TNTs occurred more frequently than false-negative. However, false-positive TNTs were not necessarily really false TNTs, since the automated method in many cases found structures that resembled TNTs, but one or both human observers had missed them in their counting. Table 1 shows the number of TNTs in each 3D image stack used for performance evaluation. The columns show the TNTs counted by both observers, the agreements between them, the number of automatically correctly classified TNTs, the false-negative and -positive, and the success rate (%).

TABLE I
Numerical results from detection of TNTs

| | Observer | Observ- | 1 and 2 | Agreeing | | | |
|---|---|---|---|---|---|---|---|
| | 1 | er 2 | agree- | automated | False | False | Success |
| Stack | count | count | ments | count | neg. | pos. | rate (%) |
| 112 | 3 | 4 | 3 | 2 | 1 | 2 | 67 |
| 113 | 4 | 3 | 3 | 2 | 1 | 5 | 67 |
| 114 | 13 | 9 | 9 | 6 | 3 | 4 | 67 |
| 115 | 9 | 7 | 7 | 5 | 2 | 2 | 71 |
| 116 | 8 | 4 | 4 | 4 | 0 | 3 | 100 |
| 117 | 5 | 4 | 3 | 3 | 0 | 2 | 100 |
| 118 | 12 | 12 | 10 | 9 | 1 | 2 | 90 |
| 119 | 13 | 10 | 10 | 4 | 6 | 5 | 40 |
| 120 | 11 | 5 | 5 | 3 | 2 | 3 | 60 |
| 121 | 10 | 7 | 7 | 5 | 2 | 3 | 71 |
| 122 | 6 | 8 | 6 | 3 | 3 | 4 | 50 |
| 123 | 2 | 2 | 0 | 0 | 0 | 1 | 100 |
| 124 | 3 | 3 | 3 | 3 | 0 | 4 | 100 |
| 125 | 5 | 4 | 4 | 2 | 2 | 1 | 50 |
| 126 | 6 | 5 | 5 | 4 | 1 | 2 | 80 |
| 127 | 6 | 6 | 5 | 5 | 0 | 2 | 100 |
| 128 | 4 | 2 | 1 | 1 | 0 | 5 | 100 |
| 129 | 1 | 1 | 1 | 0 | 1 | 4 | 0 |
| 130 | 3 | 4 | 3 | 2 | 1 | 0 | 67 |
| 131 | 4 | 4 | 4 | 3 | 1 | 1 | 75 |
| 132 | 4 | 5 | 4 | 3 | 1 | 3 | 75 |
| 133 | 4 | 2 | 2 | 0 | 2 | 1 | 0 |
| 134 | 7 | 6 | 5 | 4 | 1 | 1 | 80 |
| 135 | 8 | 6 | 6 | 4 | 2 | 2 | 67 |
| 136 | 5 | 4 | 3 | 3 | 0 | 3 | 100 |
| 137 | 3 | 3 | 3 | 2 | 1 | 3 | 67 |
| 138 | 9 | 8 | 8 | 5 | 3 | 3 | 62 |
| 139 | 12 | 13 | 9 | 7 | 2 | 6 | 78 |
| 140 | 10 | 8 | 8 | 3 | 5 | 0 | 37 |
| 141 | 3 | 3 | 1 | 1 | 0 | 0 | 100 |
| 142 | 12 | 14 | 12 | 7 | 5 | 4 | 58 |
| 143 | 6 | 6 | 5 | 2 | 3 | 3 | 40 |
| 144 | 8 | 4 | 6 | 3 | 3 | 3 | 50 |
| 145 | 8 | 11 | 8 | 6 | 2 | 6 | 75 |
| 146 | 8 | 7 | 7 | 5 | 2 | 4 | 71 |
| 147 | 9 | 8 | 7 | 4 | 3 | 4 | 57 |
| 148 | 5 | 5 | 4 | 2 | 2 | 2 | 50 |
| 149 | 7 | 6 | 6 | 3 | 3 | 1 | 50 |
| 150 | 8 | 11 | 8 | 3 | 5 | 4 | 37 |
| 151 | 4 | 3 | 3 | 3 | 0 | 2 | 100 |
| 152 | 2 | 2 | 2 | 2 | 0 | 1 | 100 |
| 153 | 8 | 8 | 8 | 6 | 2 | 3 | 75 |
| 154 | 5 | 5 | 3 | 1 | 2 | 0 | 33 |
| 155 | 3 | 2 | 2 | 2 | 0 | 3 | 100 |
| 156 | 10 | 11 | 9 | 6 | 3 | 2 | 67 |
| 157 | 8 | 8 | 8 | 6 | 2 | 3 | 75 |
| 158 | 7 | 5 | 5 | 4 | 1 | 3 | 80 |
| 159 | 8 | 10 | 8 | 5 | 3 | 4 | 62 |
| 160 | 8 | 8 | 7 | 5 | 2 | 4 | 71 |
| 161 | 7 | 7 | 6 | 5 | 1 | 4 | 83 |
| 162 | 9 | 9 | 9 | 5 | 4 | 3 | 56 |
| Total | 343 | 312 | 275 | 183 | 92 | 140 | 67 |

The last row in Table 1 displays the overall results; the total number of TNTs counted by each of the two observers and their agreements, the number of automatically correctly classified TNTs, the percentage false-negative, the percentage false-positive and the final mean success rate. The final mean success rate has been calculated as the rate between “Agreeing automated counts” and “1 and 2 agreements”. The “ground truth”, taken as agreement between two human observers, needs some justification. In such challenging and demanding image processing problems as TNT detection, a true solution is hard to achieve. Still, a trained human eye is probably the best tool available to establish a gold standard. For the current TNT detection experiment, a one-way ANOVA analysis reveals no significant difference (p=0.24) of mean TNT counts (μ1=6.7, μ2=6.1, μₐ=6.3) across all 51 stacks obtained by observer 1, observer 2, and the automated method, respectively. The count for the automated method was obtained by adding “Agreeing automated count” and “False positive”. On the other hand, the two human observers turned out to correlate more to each other than to the automated method. Pearson correlation coefficient applied to the observations of the two human observers and the automated method showed a significant correlation (α=0.05) between the two human observers (p<0.0001), in contrast to non-significant correlations between the automated method and each of the observers (p=0.42 and p=0.17). This finding justifies using the decisions by human observers as “ground truth”, since our independent observers have a high level of agreement.

TNT detection is more likely to fail in the cases where the cells are clustered, because of irregularities. Consequently we aimed at creating cell images where cells had been grown on specified patterns [Rustom A et al., BioTechniques 2000; 28:722-730], thus improving the bioinformatical ability to locate TNTs. In rare cases extremely long TNTs appear, and others may connect more than two cells. These unusual properties of TNTs seem to be connected to the type of cells being imaged.

From our TNT evaluation experiments, TNT detection is more likely to fail in the cases where the cells have close proximity or show large irregularities. An example of such typical irregularities is demonstrated in FIG. 13, where high intensity structures and sharp edges of filopodia-like structures (FIG. 13, arrows) are crossing between cells, misleading the automated detection.

The presence of these edges satisfy the TNT criteria used for the automated detection. The digital data sets also allow further statistical measures of properties of TNTs like length histogram, number of TNTs connections per cell and their slope inside the stack. To illustrate the power of the automated evaluation, we have performed measurements of length for each TNT. A 3D reconstruction of the TNTs was possible for length calculations since the algorithm keeps record of the projection range for each TNT candidate at all steps of the processing chain. The length statistics was obtained using the maximum Euclidean distance between all pixels in the TNT, adjusted for the voxel anisotropy. Integration in space was redundant since TNTs always appear as straight lines. The distribution of TNT length in our sample is illustrated in FIG. 14, statistics which is not feasible to obtain by manual methods. The length distribution of TNTs indicate that there is a high frequency of short TNTs between 1 μm and 4 μm. This may suggest that there is an optimal distance between cells for TNT formation.

### D. 3D Segmentation after Applying a Ridge Enhancing Curvature Depending Filter to the Surface Stained Image

### D1. General Principles and State of the Art

A preferred embodiment of the invention comprises further a method for segmentation of surface stained cells using ridge enhancement and morphological operators as filling and watershed segmentation. We also propose a variant of the region differencing approach for segmentation evaluation.

RIDGE ENHANCEMENT Microscopic cell images are frequently of insufficient quality for image processing purposes, and a well suited filtering will often promote a more reliable segmentation. The boundaries of a surface stained cell are outlined by ridges, thus it is reasonable to perform a ridge enhancement prior to the segmentation. Ridge detection is a well-known research field of image processing, and methods already exist to enhance the ridges of an image. The Gabour filter is a well known approach to filter fingerprint images and for extraction of important ridges [Ross A et al in Proceedings of International Conference on Pattern Recognition (ICPR); 2002]. The eigenvalue decomposition of the Hessian matrix [Frangi A F et al., Medical Image Computing and Computer-Assisted Intervention 1998; 1496:130-137; Eberly D et al., J Math Imaging V is 1994; 4(4):353-373] has been used for similar purposes. Our method for ridge enhancement is based on a curvature formulation, inspired by the eigenvalue decomposition of the Hessian matrix.

SEGMENTATION Watershed segmentation is well suited for cell segmentation. Bengtsson E et al. (Pattern Recognition and Image Analysis 2004; 14:157-167) used a watershed segmentation with double thresholds for segmentation of CHO cells stained with calcein, obtaining a success rate of between 89% and 97%. After removal of the least cell-like objects, the success rate increased, thus explaining the large range of their success rate. They applied a labeling method to measure the amount of over- and under-segmented objects, but they were not able to measure the segmentation quality of the border lines between the watershed regions. Adiga et al [Microscopy Research and Technique 2003; 54(4):260-270] used the watershed algorithm for segmentation of cell nuclei and an active surface model for further refinement to obtain an integrated segmentation approach. The author used the relative difference of volumes between the manual and the automated segmented regions to create a shape factor measuring the quality of the boundaries. A success rate at about 95% was obtained for the shape factor, however, only 11 cells were included into this statistics. There were no detailed explanation of how under- and over-segmented cells affected the shape factor, nor whether such cells were discarded. The problem of under- and over-segmentation of cells is normally less for nuclei stained cells than for surface- or cytoplasm-stained cells, because nuclei stained cells directly estimate the number of cells and the location of there nuclei, information that can be used to define markers for the watershed segmentation. The PhD thesis of Lindblad [Cytometry; 2002] offers a structured and comprehensive view of the field of cell segmentation.

EVALUATION A favorable measure of the automated segmentation is important when the quality of different segmentation methods is compared. Unfortunately, the evaluation of cell segmentation is frequently performed using subjective intuition lacking objective considerations or common and well-founded measures. However, within the area of image segmentation, numerous studies on segmentation evaluation have been published. Zhang [a) Pattern Recognition 1996; 29:1335-1346; b) Pattern Recognition Letters 1997; 18(10):963-974.7,8] offers a survey on evaluation methods for image segmentation, dividing the evaluation methods into three groups; analytical, empirical goodness and empirical discrepancy methods. Analytical methods analyze the effectiveness of segmentation methods entirely based on their analytical principles, suffering from the fact that they are rarely able to coincide with the human perception of segmentation quality. Empirical goodness methods, also referred to as stand-alone methods, are automated evaluation methods that evaluate the segmentation based on some a priori human characterization. The empirical goodness methods are extremely useful when automated feed-back evaluation of a segmentation is needed. However, as for the analytical methods, they suffer frequently from disagreements to human perception. Unfortunately, they may easily be influenced by the principles behind the segmentation method itself, if their measure of goodness is based upon the principle of the segmentation method that has been applied. This fact limits its evaluation value on a broad range of images. The empirical discrepancy methods are mainly preferred when evaluating a segmentation method. They compare the resulting segmented image to a ground truth image or a gold standard which is considered as the true solution, made by one or more human raters. For statistical significance, a segmentation evaluation must be performed on a certain amount of data, and equally important, the data that are used for development of the algorithm must be excluded from the segmentation evaluation.

Surprisingly few of the general segmentation evaluations have been applied to cell segmentation algorithms, nevertheless some authors have included an evaluation procedure. Adiga et al. [Microscopy Research and Technique 1999; 44(1):49-68] presented a semi-automatic method for segmenting 3D cell nuclei from confocal tissue images. They performed a comparative study of visual- and automated evaluation of the FISH signal counting, and achieved a more than 90% success compared to the visual counting of the FISH signals. However, they did not present any results estimating the correctness of the automated segmented cell nuclei. Malpica et al. [Cytometry 1997; 28:289-297] used the watershed algorithm for segmentation of clustered nuclei, and report that almost 90% of the test clusters were correctly segmented in peripheral blood and bone marrow preparations. These results were obtained from counting the number of correctly classified nuclei, but the exact plasma membranes were not possible to restore because these were nuclei stained images. This demonstrates a common challenge for nuclei stained images. The number of cells is easily obtained in such images, but surface stained images are required in those cases were the exact plasma membrane for each cell has to be outlined. Generally, the demands of the researcher should determine the type of cell staining that is used.

### D2. Processing Steps in Cell Segmentation

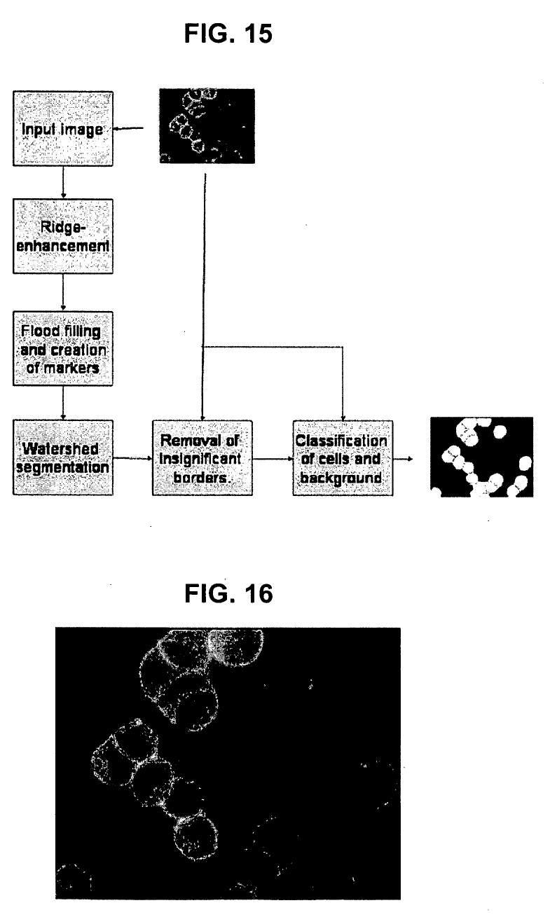

This cell segmentation procedure is designed for surface stained cells acquired by fluorescence microscopy, creating pronounced plasma membranes. The prior ridge enhancement enables a morphological flood filling which is needed to create initialization regions, also referred to as markers. These markers are then employed in the watershed segmentation to locate the plasma membranes. A watershed image is then obtained, consisting of watershed regions separated by watershed lines. The quality of each watershed line is evaluated by superimposing them on the image, and those possessing insignificant intensities compared to their surroundings are removed. Finally, the watershed regions are classified as cells and background regions. A flow scheme of the method is presented in FIG. 15. Referring to FIG. 15 the detailed processing steps of the cell segmentation using ridge enhancement are described.

### D3. Ridge Enhancement Through Curvature Filtering

The plasma membranes are expressed as ridges in surface stained images, see FIG. 16 showing surface stained PC 12 cells. Consequently, a ridge enhancing filter is applied prior the segmentation.

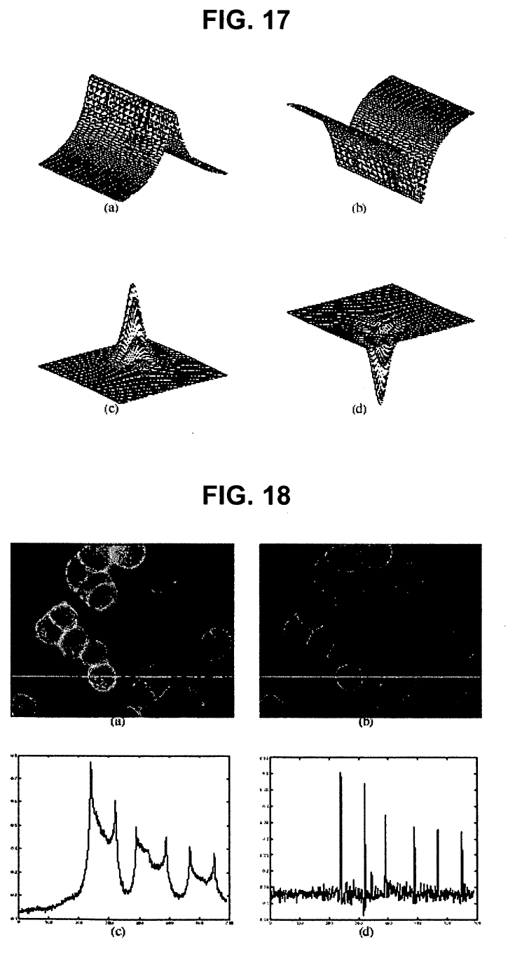

FIG. 17 shows four perfect topological variations, a ridge, a valley, a peak and a hole. Among these examples, the ridge is certainly the best model for a plasma membrane.

There are several ridge enhancing methods available. The eigenvalue decomposition of the Hessian matrix [Frangi A F et al., Medical Image Computing and Computer-Assisted Intervention 1998; 1496:130-137; Eberly D et al., J Math Imaging V is 1994; 4(4):353-373] creates an image were the ridges are nicely enhanced. However, it is a rather time consuming method tending to create artificial star-like patterns because it contains information about the second derivatives only along the main axes and the mixed derivatives. We have therefore developed another ridge enhancing filter, a method requiring less CPU time than the Hessian and one which does not create star-like patterns. A ridge is characterized by a relatively high curvature perpendicular to its pointing direction, a property which is exploited in our curvature depending ridge enhancement. The curvature κ of a 1D curve with velocity v and acceleration a is given by Finney L R, Thomas Jr in Calculus. Addison-Wesley Publishing Company, Inc; GB, 1994,

$$κ = \frac{\vert v \times a \vert}{{v}^{3}} . \qquad ( 2 )$$

which for a curve r=xi+yj is easily transformed into

$$κ = \frac{\vert {f}^{″} ( x ) \vert}{{[ 1 + {( {f}^{′} ( x ) )}^{2} ]}^{3 / 2}} \qquad ( 3 )$$

by using the transformation x=x, y=ƒ(x). Then, let ƒ (xᵢⱼ; θ) be the image values through the point xij along the direction θ. The curvature of ƒ(xᵢⱼ; θ) is calculated for each pixel in equally spaced selected directions between [0 π]. Preferably, a five-point and not a three-point derivative should be applied in the calculations of the derivatives to avoid rapid oscillations. The maximum curvature image Cₘₐₓ and the minimum curvature image Cₘᵢₙ are then calculated at each point i,j as the maximum and minimum projection of the curvatures which have been calculated between to [0 π]. The plasma membranes are characterized by a high maximum curvature, similar to the peaks. Preferably, it is advantageous to distinguish ridges from peaks. This can partly be accomplished as peaks also have a relatively high minimum curvature, in contrast to ridges which have a small minimum curvature. However, practically it is challenging to distinguish ridges from peaks as there exist no perfect shapes in natural images. The peaks are often elongated, resembling ridges, and peaks are frequently superimposed on ridges, creating ridges resembling peaks. Consequently, a removal of all peaks will create numerous gaps in the ridges, a situation which in our case in not acceptable for the further processing. To preserve all ridges, the minimum curvature image itself is therefore used as the ridge enhanced image.

### D4. Morphological Flood Filling and Creation of Markers

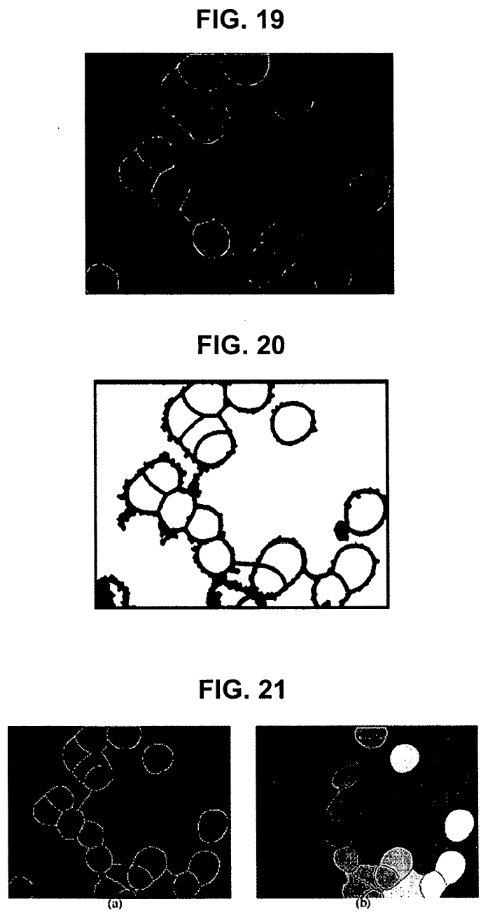

The exact plasma membranes are found by a marker controlled watershed segmentation where the markers are created by morphological flood filling. Cells in surface-stained images are characterized as closed regions with significantly higher intensities at their borders than around. Morphological flood filling [Soille Pierre. Morphological Image Analysis: Principles and Applications. Secaucus, N.J., USA: Springer-Verlag New York, Inc.; 2003] is therefore used to create internal markers inside the cells, each marker defining a separate object of interest for segmentation. All holes defined as dark pixels surrounded by lighter pixels are filled from flood filling. It is performed on the grayscale ridge-enhanced images similar to FIG. 18(b), dividing them into closed and connected regions, and replacing each pixel value by its regions mean value. In such a manner, multiple constant valued regions are created, and they are easily detected by their zero gradient. Further, to obtain a flood filling of the background, the image border values are raised iteratively until the background was filled by flood filling in the same manner as the cell regions. An example of such a flood filling process performed on FIG. 18(b), is shown in FIG. 19.

The constant valued regions are extracted by calculating the zero gradients and then converted into a binary image. The small and insignificant markers are removed, and after morphological closing and filling, a minima marker image is achieved, depicted in FIG. 20.

### D5. Watershed Segmentation

The markers in the minima marker image are used as initialization regions for the watershed segmentation. To save computational time, a 2D watershed segmentation as implemented in MATLABs Image Processing Toolbox [Vincent L, Soille P in IEEE Transactions on Pattern Analysis and Machine Intelligence 1991; 13(6):583-598.] is performed as a consequence of the time consuming process of creating 3D markers. Then, the watershed regions are used as markers for a 3D watershed segmentation. FIG. 21(b) shows one plane of the 3D watershed image which is then attained, comprising watershed lines (black) and the connected watershed regions labeled with increasing integers.

Then, all watershed lines are tested for their significance. They are superimposed in the original image, and the mean image intensity of each watershed line is compared to the mean image intensity on an artificial, bilateral structure following the watershed line. From thresholding, it is decided whether this is a locally high-intensity structure. If not, it is rejected as over-segmentation. A correct segmentation is more accessible from an over-segmentation than from an under-segmentation, a certain amount of over-segmentation is therefore preferred. The watershed regions are then classified into background and cells according to simple classification rules:

All convex regions below a certain size are classified as cells.

However, if a non-convex region contains internalized stained particles, it is still classified as a cell despite its shape.

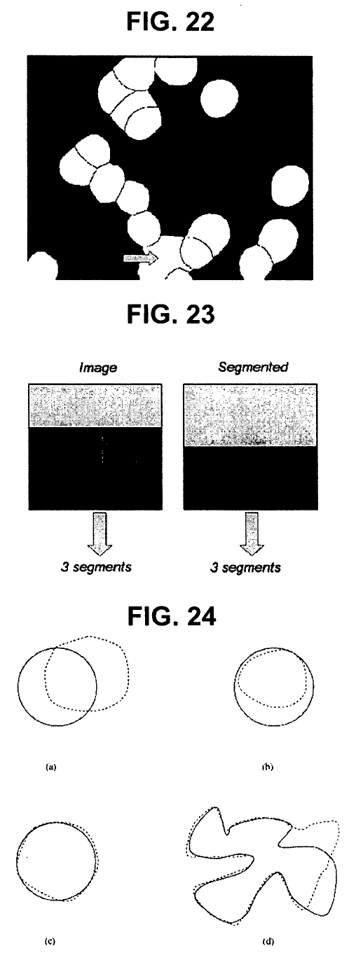

Such simple classification rules are applicable due to a previously high-quality segmentation with a minimum of over-segmentation. Classification of heavily over-segmented images is extremely challenging since the segmented regions acquire properties regarding their shape that are not reflecting the true shape of a cell. The final classification of the watershed regions in FIG. 21(b) is displayed in FIG. 22, the arrow pointing out a region which is incorrectly classified as a cell. This is a typical error that occurs because the significance test of the watershed lines failed due to an extraordinary weak cell border. The watershed line was therefore removed.

### D6. Method for Segmentation Evaluation

Segmentation evaluation in general is a well discussed problem [Zhang Y J. In Pattern Recognition 1996; 29:1335-1346; Zhang Y J in Pattern Recognition Letters 1997; 18(10):963-974]. In contrast, evaluation of cell segmentation is a rarely discussed topic. We will apply a modified empirical discrepancy method (see section 1), sometimes referred to as region differencing, to construct a framework for evaluation of cell segmentation. According to Zhang [Pattern Recognition 1996; 29:1335-1346], the empirical discrepancy methods can be divided into four classes, where the discrepancy is based on one or more of the following:
(i) The number of mis-segmented pixels.
(ii) The position of mis-segmented pixels.
(iii) The number of objects in the image.
(iv) Feature values of segmented objects.

An appropriate measure for correctness of segmentation must comprise both the number of segmented regions, equivalent to (3), and the co-localization of the area between the automated and the manually segmented regions, equivalent to (1) and (2). FIG. 23 demonstrates a synthetic image (left) and the segmentation of it (right), where (3) is fulfilled, but (1) and (2) only partly. The segmentation yields three segments, thus the number of segments is equivalent with those in the original image. Still, it is a poor segmentation because the segments are only to a certain degree co-localizing with the segments in the original image.

In our opinion, a segmentation evaluation must primarily penalize situations according to (1) and (3), but (2) and (4) can easily be included into the region differencing approach as well.

Goumeidane et al. [Pattern Recognition Letters 2003; 2(10):411-414] proposed an empirical discrepancy method that relies on the position of mis-segmented pixels (2), but excluding the features (1), (3) and (4). Still, they obtain an intuitively correct measure of differences between a segmented region and a reference region by superimposing them. Our method takes advantage of this concept by superimposing two corresponding regions, one taken from the reference segmentation and the other from the automated segmentation. The relative overlap of area between them is then measured, corresponding to (1). Further, it is desirable to design a method taking into account the requirements of (3), penalizing over- and under-segmented regions, also referred to as degeneracy. As pointed out by Unnikrishnan [Unnikrishnan R et al., in: Proceedings of the 2005 IEEE Conference on Computer Vision and Pattern Recognition (CVPR '05), Workshop on Empirical Evaluation Methods in Computer Vision; 2005], region differencing may suffer from degeneracy and lack of non-uniform penalty. Degeneracy is demonstrated by the fact that one pixel per segment or one segment for the whole image will both give zero error. A method for segmentation evaluation must also be able to deal with situations of both uniform and non-uniform penalty. A non-uniform ground-truth is desirable in the cases where multiple hand-drawn solution differ significantly, or when a high degree of reliability is needed. Our region differencing approach is able to deal with both degeneracy and uniform/non-uniform penalty.

Based on an empirical discrepancy method using the number of mis-segmented pixels and the number of objects to measure discrepancy, we want to discuss an approach in agreement with requirements (1)-(4) pointed out by Zhang [Pattern Recognition 1996; 29:1335-1346]. Conceptually, the correctness of a segmentation is well conceived by evaluating the overlap between clusters in the true solution and the automated segmentation. For our method, let the ground-truth image S\ created from visual inspection, consist of m non-connected regions {S^{t}ᵢ}. Equivalently, let the binary, automatically segmented image S comprise n non-connected regions {Dⱼ}. To include the request of non-uniform penalty, the true solution image function 0≦ƒ(S^{t})≦1 can be a function taking any value, based on the agreement between multiple human observers. A similarity matrix A^{union}: m×n with elements A^{union}ᵢⱼε[0 1] is then computed, each element containing the total intensity value of intersecting non-zero pixels between {S^{t}ᵢ} and {Sⱼ}, normalized by the total intensity value of the union between S^{t}ᵢ and Sj,

$${A}_{ij}^{union} = \frac{f ( {S}_{i}^{t} ⋂ {S}_{j} )}{f ( {S}_{i}^{t} ⋃ {S}_{j} )} . \qquad ( 4 )$$

In the case of a perfect segmentation where S^{t}ⱼ→Sj, Aij→1. Oppositely, if the segmentation is ill-behaving such that S^{t}ᵢ∩Sj=0, then Aᵢⱼ=0. Thus, the value Aᵢⱼ reflects the amount of overlap between the reference region and the segmented region, penalizing both lack of intersection between Sitr^{ue} and Sj, and over- and under-segmentation. This is the reason for our choice of A^{union} as the best similarity matrix for further processing. However, there are several possible extensions to Eq. (4). Instead of scaling the total intensity value to the union, it can be scaled to the area of the manually segmented region S,

$${A}_{ij}^{man} = \frac{f ( {S}_{i}^{t} ⋂ {S}_{j} )}{f ( {S}_{i}^{t} )} , \qquad ( 5 )$$

to the automated segmented region Sj

$${A}_{ij}^{ant} = \frac{f ( {S}_{i}^{t} ⋂ {S}_{j} )}{f ( {S}_{j} )} . \qquad ( 6 )$$

or it can be scaled to the maximum area of those two,

$${A}_{ij}^{max} = \frac{f ( {S}_{i}^{t} ⋂ {S}_{j} )}{max ( f ( {S}_{i}^{t} ) \cdot f ( {S}_{j} ) )} . \qquad ( 7 )$$

Eq. 5 and Eq. 6 are capable of distinguishing between under- and over-segmentation, respectively. Eq. 7 is a good measure if there are large alternating variations between over- and under-segmentation.

A selection of synthetic examples are shown in FIG. 24, displaying how the similarity measure is able to deal with divergent situations. The area inside the solid lines is the reference solution, and the area inside the dotted lines is the automatically segmented area.

Table 2 contains the corresponding parameters for the segmentation evaluation of FIG. 24, where increasing values from 0->1 correlate with an improved segmentation. In (a), the similarity measure A^{union}=0.35, thus the area inside the dotted line is a bad representation of the area within the solid line. In (b), the similarity measure A^{union}=0.63, somewhat higher than in (a) due to the lack of over-segmentation, (c) represents a good segmentation with A^{union}=0.91, in agreement with human perception. The segmentation of (d) is distorted in the right part of the image, resulting in a fairly acceptable similarity value of A^{union}=0.75.

TABLE 2
Segmentation evaluation parameters from the images
in FIG. 24. Example (c) acquires the highest score, close to 1.

| | A^{union} | Evaluation |
|---|---|---|
| (a) | 0.35 | Poor |
| (b) | 0.63 | Poor |
| (c) | 0.91 | Good |
| (d) | 0.75 | Medium |

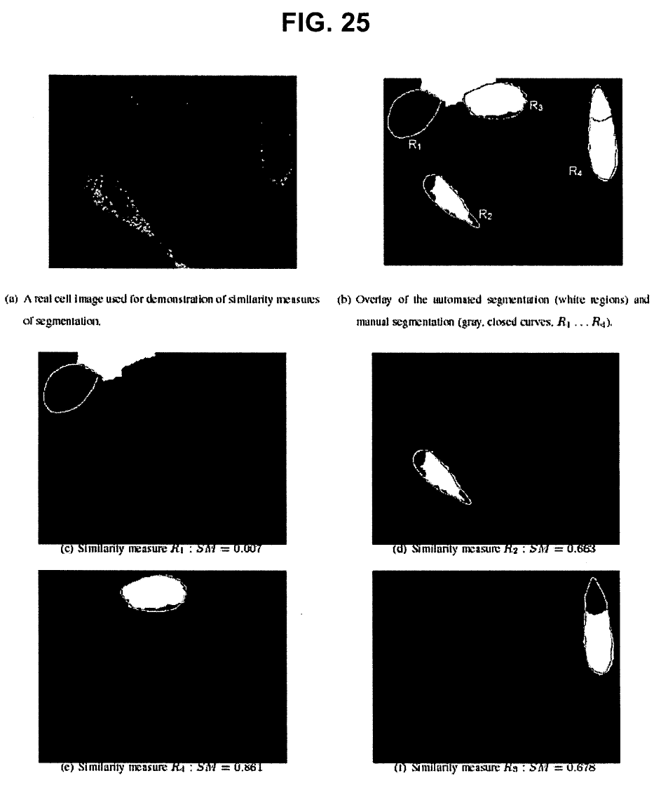

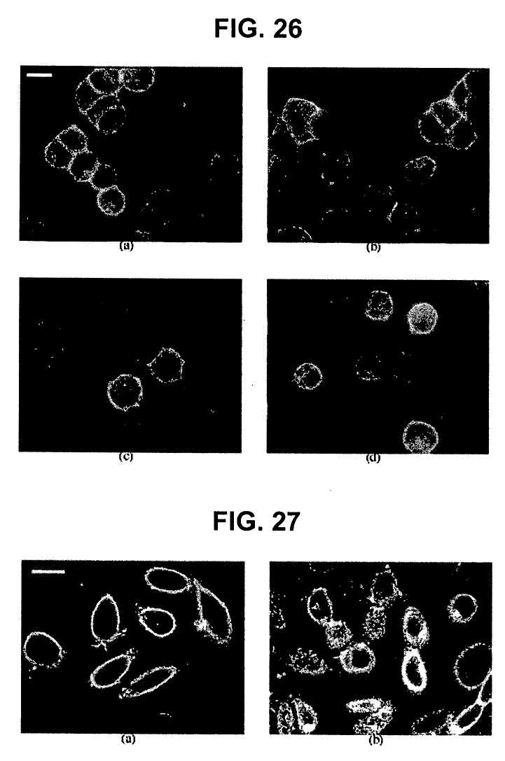

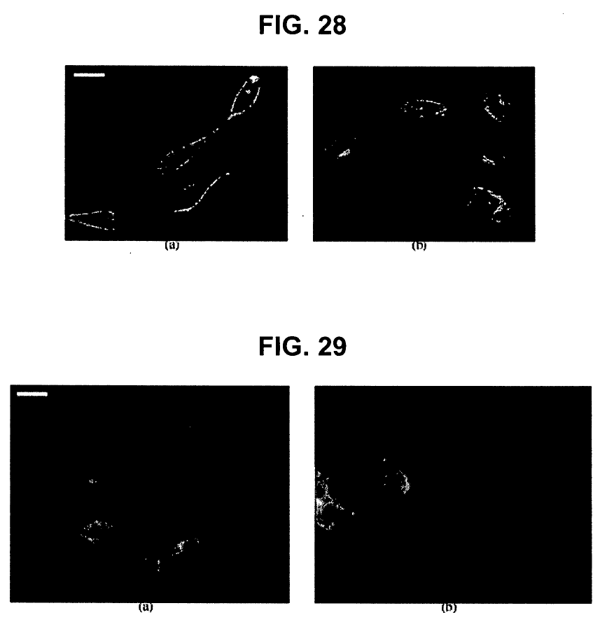

FIG. 25 displays automated segmented regions (white) and the ground-truth (gray borders) with the corresponding similarity measures, taken from a real cell image. These measures are inserted into the similarity matrix A^{union}, each row corresponding to a single region from the ground truth image (FIG. 26)

To properly deal with the problem of degeneracy, two important assumptions must be made. First, each automated segmented region must represent one and only one manually segmented region, and vice versa.

This is equivalent to A^{union} containing at most one non-zero value per row and column. Therefore, the matrix A^{union} as a whole must contain no more than N non-zero values, N=min(m, n). This feature is accomplished by iterating through the elements in A^{union} according to decreasing values, at each iteration removing the element if there exists a larger value in the same row or column. If not, the element remains unchanged. This optimizing problem can be formulated mathematically as the elements in A=A^{union} maximizing a matrix norm i.e. the Frobenius norm defined as

$${‖ A ‖}_{F} = \sqrt{{\sum }_{ij} {\vert {a}_{ij} \vert}^{2}} . \qquad ( 8 )$$

under the constraints K^{r}={Kir} and K^{c}={K^{c}}

$$\begin{matrix}{K}_{i}^{r} = {\sum }_{j} H \vert {a}_{ij} \vert \leq 1 ∀ i = \{ 1 … m \} and \\ {K}_{j}^{c} = H \vert {a}_{ij} \vert \leq 1 ∀ j = \{ 1 … n \} . \end{matrix} \qquad ( 9 )$$

where H(x) is the heaviside function. The constraints will ensure a maximum number of one non-zero element for each row and column. The iterations are performed in decreasing order through all matrix elements of A, for each iteration removing the element if the constraint is violated. Then, by definition, the largest possible Frobenius norm of A is obtained after the iterations have been through all elements in A. The MATLAB code for calculating this matrix can be viewed in the Appendix.

$$SM = [ \begin{matrix} & 0 & 0 & 0 & 0 & 0 & 0 \\ 0 & & 0 & 0 & 0 & 0 & 0 \\ 0.003 & 0 & & 0 & 0 & 0 & 0 \\ 0 & 0 & 0 & 0 & & 0.239 & 0\end{matrix} ] \begin{matrix}\to {R}_{1} \\ \to {R}_{2} \\ \to {R}_{3} \\ \to {R}_{4}\end{matrix} \qquad ( 10 )$$

Eq 10: The similarity matrix for the segmentation of FIG. 11(b), equivalent to FIG. 11(c-f). R3 and R4 can each be represented by two different automated segmented regions, but the encircled values are chosen since they optimize the Frobenius norm for A^{union}.

Under-segmentation will create blank rows in the similarity matrix A^{union}, and over-segmentation will create blank columns, see Eq. 11 to visualize the effects of over- and under-segmentation on A^{union}.

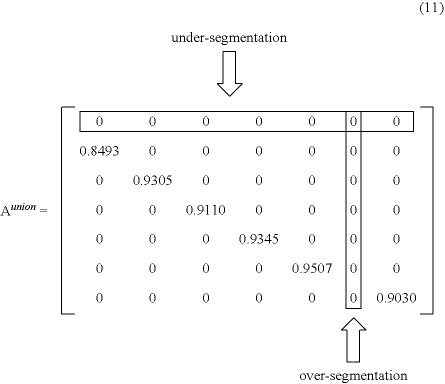

Eq. 11: The similarity matrix A^{union} after optimizing the Frobenius norm. The elements range from 0→1, increasing with the quality of the segmentation. The vertical frame demonstrates over-segmentation where an automated segmented region is unable to represent any manually segmented region. Oppositely, the horizontal frame demonstrates under-segmentation where a manual segmented region is not well represented by any of the automated segmented regions.

The overall segmentation measure SM for the image is obtained from summing all elements in the similarity matrix, after each of them have been scaled to the number of pixels in the manual region they are related to. This scaling is performed in order to ensure that each manual segmented region will influence the final similarity measure in a way which is closely related its area relative to the total manual segmented area in the image. Thus, large regions will influence SM more than small regions. The final similarity measure SM is calculated as the sum of a scaled to the relative number of pixels in each region Nᵢ,

$$SM = {\sum }_{i} {a}_{ij}^{union} \frac{{N}_{i}}{N} , \qquad ( 12 )$$

where N is the total number of pixels in the manual segmented image, N=ΣᵢNᵢ. After these operations, SM is still a number in [0 1] where a value close to 0 relates to a poor segmentation, and a value close to 1 labels an excellent segmentation.

### D7. Results

Our segmentation algorithm is a versatile method, designed to segment cells with a pronounced cell border. For such images, the algorithm can distinguish between single cells as well as touching cells. It has a broad range of applications, which is demonstrated in the following sections were two different cell types, two different stainings and three different microscopes are used to evaluate the segmentation algorithm. The cells in these experiments share the features of distinct and well-marked cell borders. Five experiments showing the effectiveness of the segmentation method are presented in the following order
(i) Segmentation of WGA stained PC 12 cells from wide-field imaging.
(ii) Segmentation of WGA stained NRK cells from a spinning disc.
(iii) Segmentation of WGA stained NRK cells from a confocal microscope.
(iv) Segmentation of f-EGFP stained PC 12 cells from wide-field imaging.
(v) Segmentation of WGA stained cells from wide-field imaging where cell division is inhibited.

Experiment 1-4 are evaluated using the similarity measure SM described in the previous section, where a hand-drawn solution is taken as ground truth. The last experiment was performed in order to investigate whether the program could detect that cells treated with thymidine will increase size in comparison to a control group.

All code in this paper was implemented in MATLAB and the experiments were carried out on a Linux workstation running a 2.4 GHz AMD processor. To avoid over-fitting to data, the method was developed on a separate data set not used for the final evaluation. The segmentation program was executed using 3D image stacks, however, the human evaluation was achieved from one 2D plane extracted from the middle of each image stack. This extraction was performed to save human time as it was considered more valuable creating multiple 2D images containing the ground truth, rather than fewer 3D stacks. To fit the 3D automated segmentation to the 2D hand-drawn solution, the middle plane from the automated segmentation was extracted and compared to the hand-drawn solution.

### D8. Segmentation of WGA Stained PC12 Cells

A set of 10 stacks containing WGA stained PC12 cells were in this example used to evaluate the segmentation algorithm, see above for the preparation of the images. The input images as they apply are presented in FIG. 26, showing cell cultures of PC 12 cells stained with WGA. The images exhibit large variations of their illumination and the shape and number of cells. The diameter of the PC 12 cells vary roughly between 10 and 15 micrometers. The images are afflicted with Gaussian noise in addition to internalization of stained particles. These particles appear as light spots inside the cells, creating strong edges that are easily mistaken as cell borders by the automated method. Especially challenging situations arise where the plasma membrane of a cell is not continuously stained, manifesting itself as a fractured ridge.

The 2D manual ground truth contained 163 cells, and Table 6 shows the output from the segmentation evaluation using the similarity measure SM described above. The overall success rate for the entire experiment, the lowest row in Table 6, has been adjusted for the number of manually segmented cells in each image. We obtained, on an overall, a success rate of SMᵤₙᵢₒₙ=93.9%, a result which is very comfortable. SMₘₐₙ and SMₐᵤₜ, are approximately equal, thus the amount of under-segmentation (100%-95.3%=4.7%) and over-segmentation (100%-96.1%=3.9%) was in the same order, around 4%.

TABLE 3
Numerical results from automated detection of PC 12 cells.
The segmentation algorithm obtained
a success rate of SMᵤₙᵢₒₙ = 93.9%.

| Stack | N_{cells} manually | SMₘₐₙ (%) | SMₐᵤₜ (%) | SMₘₐₓ (%) | SMᵤₙᵢₒₙ (%) |
|---|---|---|---|---|---|
| 1 | 19 | 97.3 | 97.8 | 96.9 | 95.9 |
| 2 | 23 | 97.8 | 97.4 | 96.8 | 95.8 |
| 3 | 13 | 97.7 | 98.2 | 97.7 | 96.8 |
| 4 | 12 | 97.2 | 97.3 | 96.6 | 95.5 |
| 5 | 18 | 97.5 | 98.7 | 97.3 | 96.3 |
| 6 | 22 | 89.1 | 90.0 | 88.9 | 88.0 |
| 7 | 21 | 90.1 | 91.3 | 89.8 | 88.7 |
| 8 | 8 | 97.2 | 98.7 | 96.9 | 95.9 |
| 9 | 14 | 97.2 | 98.0 | 96.7 | 95.3 |
| 10 | 13 | 97.2 | 98.4 | 96.8 | 95.6 |
| Total | 163 | 95.3 | 96.1 | 95.0 | 93.9 |

D9. Segmentation of WGA Stained NRK Cells from a Spinning Disc

NRK cells stained with WGA were imaged using a spinning disc confocal as described above. Two representative images are shown in FIG. 27. Similar to the WGA stained PC 12 cells, the cell borders are clearly marked, although the image contains a substantial amount of noise.

The segmentation was performed in 2D as a consequence of the large inter-plane distances, creating a more complex situation for a 3D segmentation. The plane chosen for segmentation was taken from above the filopodia level, since the filopodia are long and thin-like structures, requiring a different segmentation method than the watershed segmentation which was used in this project. The data set contained 137 manually segmented cells. The segmentation evaluation revealed success rate of SMᵤₙᵢₒₙ=81.5% (Table 4) which is satisfying for most applications. It was also capable of estimating a higher false negative (100%−84.1%=15.9%) than false positive rate (100−91%%=9.0%).

TABLE 4
Numerical results from segmentation of WGA stained NRK cells
imaged on a spinning disc.

| | | | | | SMᵤₙᵢₒₙ |
|---|---|---|---|---|---|
| Stack | N_{cells} manually | SMₘₐₙ (%) | SMₐᵤₜ (%) | SMₘₐₓ (%) | (%) |
| 1 | 8 | 88.0 | 93.6 | 87.9 | 86.6 |
| 2 | 7 | 82.5 | 85.1 | 82.2 | 80.0 |
| 3 | 5 | 95.8 | 99.0 | 95.8 | 93.8 |
| 4 | 14 | 96.9 | 95.3 | 94.9 | 92.6 |
| 5 | 5 | 94.9 | 98.4 | 94.8 | 93.4 |
| 6 | 7 | 83.2 | 87.9 | 82.8 | 80.6 |
| 7 | 6 | 95.2 | 97.6 | 94.7 | 93.0 |
| 8 | 3 | 71.8 | 73.8 | 71.8 | 70.7 |
| 9 | 3 | 95.1 | 95.7 | 94.4 | 91.2 |
| 10 | 3 | 91.7 | 96.8 | 91.7 | 89.0 |
| 11 | 3 | 93.6 | 98.0 | 93.6 | 91.8 |
| 12 | 6 | 63.8 | 79.5 | 63.8 | 62.2 |
| 13 | 7 | 88.5 | 98.3 | 88.5 | 87.2 |
| 14 | 9 | 64.4 | 79.1 | 64.4 | 62.8 |
| 15 | 11 | 91.9 | 98.0 | 90.9 | 89.4 |
| 16 | 8 | 78.4 | 81.5 | 77.6 | 75.5 |
| 17 | 11 | 80.2 | 88.4 | 80.2 | 78.3 |
| 18 | 14 | 76.5 | 88.1 | 73.0 | 71.5 |
| 19 | 7 | 77.2 | 98.2 | 77.2 | 75.9 |
| Total | 137 | 84.1 | 91.0 | 83.3 | 81.5 |

A similarity measure of SMᵤₙᵢₒₙ=81.5% was obtained, acceptable for most applications

This experiment was conducted on WGA stained NRK cells which were imaged on a confocal microscope, resulting in stacks of 14 planes each. A single plane from each stack was extracted and used for segmentation. The images are of poorer segmentation quality than the those from the spinning disc, with a higher degree of fragmentation of the plasma membranes, creating oscillations. The ground truth was made by a human rater, and the ground truth was compared to the automated solution using the similarity measure described above. The results from the segmentation evaluation are shown in Table 5, SMᵤₙᵢₒₙ=74.1% which is acceptable for most applications. The similarity measure SMₐᵤₜ obtained a very high value of 93.4%, implying a low degree of over-segmentation, (100%-93.5%)=6.5%.

TABLE 5
Numerical results from segmentation of NRK cells
imaged at a confocal microscope

| | | | | | SMᵤₙᵢₒₙ |
|---|---|---|---|---|---|
| Stack | Ncells_{manually} | SMₘₐₙ (%) | SMₐᵤₜ (%) | SMₘₐₓ (%) | (%) |
| 1 | 4 | 82.6 | 97.9 | 82.6 | 80.2 |
| 2 | 2 | 66.3 | 98.1 | 66.3 | 65.0 |
| 3 | 2 | 69.1 | 100.0 | 69.1 | 68.6 |
| 4 | 4 | 76.5 | 98.0 | 75.6 | 73.9 |
| 5 | 4 | 82.3 | 99.4 | 82.3 | 81.4 |
| 6 | 5 | 92.7 | 99.3 | 92.6 | 89.2 |
| 7 | 5 | 90.4 | 98.9 | 89.7 | 87.9 |
| 8 | 8 | 70.2 | 97.9 | 70.2 | 68.4 |
| 9 | 6 | 70.9 | 86.4 | 69.2 | 65.6 |
| 10 | 7 | 78.6 | 97.7 | 78.6 | 76.7 |
| 11 | 6 | 50.7 | 62.6 | 50.3 | 48.7 |
| 12 | 8 | 86.9 | 95.7 | 86.3 | 81.7 |
| Total | 61 | 76.9 | 93.4 | 76.5 | 74.1 |

An overall success rate of SMᵤₙᵢₒₙ=74.1% is obtained.

D11. Segmentation of f-EGFP Stained PC12 Cells from Wide Field Imaging

This experiment was conducted to exemplify an extremely difficult situation for segmentation. PC12 cells were stained as described above. The images are afflicted by large drop-out of cell membranes and represent therefore a particularly challenging task for cell segmentation, manually as well as automatically. Especially note the significant drop-out of cell membranes in FIG. 29(a). The drop-out of cell membranes occurs due to an uneven staining of the cell membranes, and because of different metabolism of the dye between cells and between inter-cellular regions.

The segmentation evaluation reveals a significant lower success rate (SMᵤₙᵢₒₙ=41.6%) than for the WGA stained PC 12 cells (SMᵤₙᵢₒₙ=93.9%) described above. This result is due to the large drop-out of the cell membranes. Still, SMₐᵤₜ=58.7% is a fairly acceptable value, and compared to SMₘₐₙ=42.7% indicating that the majority of the segmentation errors were caused by under-segmentation.

TABLE 6
Numerical results from segmentation of f-EGFP stained PC 12 cells.

| | | | | | SMᵤₙᵢₒₙ |
|---|---|---|---|---|---|
| Stack | N_{cells} manually | SMₘₐₙ (%) | SMₐᵤₜ (%) | SMₘₐₓ (%) | (%) |
| 1 | 6 | 62.9 | 11A | 60.1 | 58.8 |
| 2 | 5 | 49.3 | 79.9 | 49.3 | 49.1 |
| 3 | 6 | 79.4 | 97.7 | 79.4 | 77.6 |
| 4 | 1 | 34.9 | 99.8 | 34.9 | 34.8 |
| 5 | 4 | 84.7 | 98.4 | 84.7 | 82.9 |
| 6 | 7 | 26.3 | 29.5 | 26.3 | 26.0 |
| 7 | 5 | 0.0 | 0.0 | 0.0 | 0.0 |
| 8 | 4 | 0.0 | 0.0 | 0.0 | 0.0 |
| 9 | 2 | 25.4 | 100.0 | 25.4 | 25.4 |
| Total | 40 | 42.7 | 58.7 | 42.3 | 41.6 |

Due to the complex images, the segmentation evaluation reveals a significant lower success rate (SMᵤₙᵢₒₙ=41.6%) than for the previous experiments

D12. Segmentation of WGA Stained PC12 Cells Treated with Thymidine

This experiment was performed to validate the segmentation algorithm by taking advantage of a biological known effect. It is an established fact that cell division is inhibited in cells treated with thymidine, causing larger cells. The purpose was to check whether the segmentation algorithm would be able to detect the increased size of these cells. The PC 12 cells were prepared according to the description in Section 3.1, and then divided into two groups. One group was used as a control, and the other group was exposed to thymidine. The biological experiment was conducted three times, and the segmentation was performed in 3D. The segmentation was blind, as the person executing the segmentation had no information available concerning which of the two groups were treated with thymidine. Three parameters measuring size were calculated for the regions: the volume (v), the major-(Dₘₐⱼ) and the minor axis length (Dₘᵢₙ). The major and the minor axis lengths are defined as the length of the major and minor axis of the ellipse having the same normalized second central moment as the region. The major and minor axis length were calculated in 2D for the mid plane, and the volume was calculated in 3D. Table 7 displays the results from the two-tailed t-test of the segmentation. The first two columns show the number of cells in the treated and untreated group. For all three experiments, the p-values describing the difference in volume, major- and minor axis length were computed (column 4-6). There was a significant difference (α=0.05) for the investigated properties in all experiments, except from the minor axis length (column four) which was not significant in the first experiment. Still, we consider it proven that the mean size of a cell treated with thymidine will increase compared to a control group. The sample mean values of the major- and minor axis lengths followed by the standard error of the mean are shown in columns six to ten, stating that the mean diameter of an untreated PC12 cell can approximately vary between 8 μm and 15 μm.

TABLE 7
Numerical results from three experiments of cells treated with tymidine.

| | N_{cells} (+) | N_{cellss} (−) | pv | pmaj | Pmin | Dₘₐj (+) | Dₘₐⱼ (−) | Dₘᵢₙ (+) | Dₘᵢₙ (−) |
|---|---|---|---|---|---|---|---|---|---|
| Exp. 1 | 111 | 156 | .005 | .060 | .009 | 15.26 + 0.40 | 14.32 + 0.31 | 9.98 + 0.32 | 8.95 + 0.24 |
| Exp. 2 | 288 | 333 | io−⁷ | io−⁷ | 0.001 | 16.79 + 0.32 | 14.65 + 0.25 | 9.21 + 0.19 | 8.42 + 0.16 |
| Exp. 3 | 306 | 356 | .003 | .002 | .091 | 15.83 + 0.25 | 14.80 + 0.21 | 9.01 + 0.18 | 8.62 + 0.15 |

The first two columns show the number of cells in the treated group (+) and the control group (−). A two-tailed t-test comparing the size between the cells in two groups was computed, and the p-values for the volume (pᵥ), the major axis length (pₘₐⱼ) and the minor axis length (pₘᵢₙ) is shown in column (3-5). Finally, the mean major- and minor axis lengths for the two groups is given in μm, D SEM (Standard Error of the Mean).

### D13. Conclusion

A ridge enhancing filter was necessary to enhance the ridges, which are the image features that characterize the plasma membranes. Based on this filter, a morphological flood-filling operation was performed, thus creating internal markers of the cells, ideally one per cell. These markers were then used as initialization regions for a watershed segmentation, outlining the plasma membranes. Due to a certain over-segmentation, the watershed lines marking the borders between the segmented regions had to undergo an evaluation process to determine whether they ought to be removed or not. Finally, the segmented regions were classified into cells and background according to some simple classification rules. The cell segmentation tool was compared to a manually segmented data-set. The correctness evaluation was performed using a region differencing variant, calculating the overlap between a segmented region and all automated regions. Two relative correctness measures were then obtained, one from scaling the area of overlap to the area of the manually segmented region, and one from scaling it to the automatically segmented regions. The segmentation was considered to be good for a specific region if there existed a good value for both measures.

We obtained, using this variant of the region differencing approach, a higher success rate. The two different success rates were achieved from either using an area depending scaling or not. The highest success rate was obtained if the importance of the cell was scaled according to its size, to a certain amount disregarding the smallest cells. The automated segmentation tool was also used to demonstrate its usefulness by calculating selected statistical parameters for a large amount of PC 12 cells. Such cell segmentation tools are highly demanded in biology because of their effectiveness and objectivity, properties that humans lack.

### CONCLUSIONS

Automated methods are increasingly important in cytometry for cell counting and characterization. High-throughput statistics can be obtained from automated cell segmentation, which is useful for quantification of cellular systems. This application presents a method for segmentation of surface stained PC12 cells in fluorescence images.

In summary, the examples show that the method for automated cell analysis, cell classification and/or determination of transport and communication between living cells is working and can be used in industry for a quantified testing of drugs and physical therapies on cells. The automated detection also allows estimation of statistical information on selected properties of TNTs in addition to counts. One important parameter would be to know how many TNT connections a cell is generating. This parameter might vary according to different biological conditions as they occur during pathological processes. Provided that TNTs are involved in certain pathological states of multicellular organisms, it can be of great value to either block or enhance their function. In this respect, the screening of drugs for modulating TNT formation and function benefit from this automated method for quantitative analysis of TNTs. In this way the effect of drugs could be evaluated by high throughput screening.

Using our method for automated finding of TNTs and connecting cells in two-channel fluorescent images of cultured cells, we obtained an overwhelming success rate of more than 90% using manual labeling as gold-standard. The success rate of the TNT detection depends critically on proper classification of cells and background. This part has been accomplished by using a biological cell marker image in combination with image processing techniques. Furthermore, a proper detection of TNTs also depends on cell cultures with optimal and reproducible growth conditions. Under normal cell culture conditions, cells often grow in close proximity which makes it difficult to detect TNTs. This problem has been illustrated. To circumvent this problem, cells should be grown on specific matrix patterns [Arnold M et al., ChemPhysChem 2004; 5(3):383-388] which guarantee more standardized cell culture conditions, in particular, when ensuring a certain distance between cells, and thus improving the methods ability to locate TNTs.

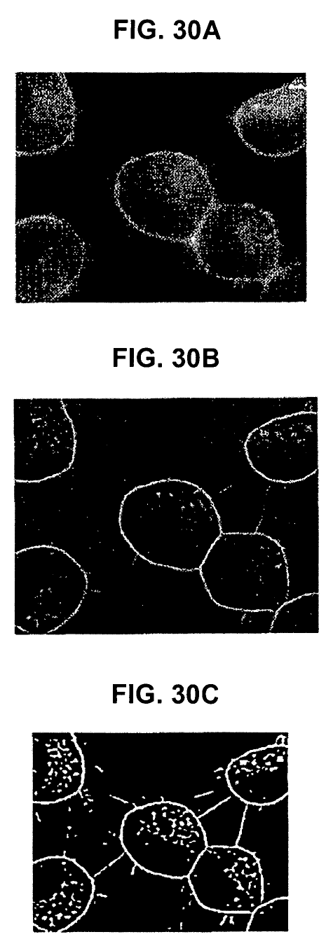

In the base method, we apply Canny's edge detector and watershed segmentation of 2-D projections for locating TNTs. The cell borders are obtained using marker controlled watershed segmentation, where the degree of segmentation is determined by flood filling imposed markers for the segmentation. The segmented regions are classified into cells and background based on a second image channel, a biological cell tracker. The TNTs then appear as structures crossing background while connecting two different cells at their nearest distance. The success rate of the TNT detection depends upon a high reliability on the part for classification of the watershed regions into cells and background. A success rate of more than 90% can be obtained by a variant of the region differencing approach for segmentation evaluation. This variant method comprises the application of a new ridge enhancing curvature filter to the surface stained images to enhance the plasma membranes. In an alternative approach, ridge enhance is applied to the image and then followed by an adaptive thresholding. After ridge enhancement, a substantial amount of noise has been removed, and it is possible to apply a local adaptive threshold method to find the TNTs. The adaptive threshold method converts the ridge enhanced image into a binary image containing significant, high intensity structures. This process is exemplified in FIG. 30, where the ridge-enhanced image has been converted into a binary image. The adaptive threshold method used the Gaussian blurred image itself as the threshold, thus creating a local threshold in each pixel, robust against uneven illumination of the image. All structures inside cell regions are discarded and the rest are skeletonized to simplify further processing. All other steps follow as described above.

Future work will include time series of 3-D image stacks, as well as examination of the dynamical formation and degradation of TNTs.

1. Method for automated cell analysis, cell classification and/or determination of transport and communication between living cells, comprising the steps of: singularizing cells in a culture medium and spreading or plating cells in a monolayer onto a substrate for a predetermined period; staining the cells with a fluorescent or luminescent dye, immunofluorescence or other detectable microscopic stain to obtain stained plasma membranes, TNTs, flagella and/or other cell particles for 3-D cell microscopy; performing image acquisition in multiple focal planes; analysing the images of the multiple focal planes as to the staining intensity over background in predetermined volumes; segmenting structures into regions and classifying the regions as to shape, curvature and other selected properties; selecting structures that are candidates for TNTs or flagellae based on the property that a TNT or a flagella must cross background; reducing the number of candidates for TNTs or flagellae by keeping or, in the case of flagellae, rejecting those crossing from one cell to another.

2. Method of claim 1, comprising a staining of the cells with at least two different cell dyes, one of which staining the cytoplasm.

3. Method according to claim 1 or claim 2, comprising a staining of the cells with at least two different cells dyes, one of which displaying cell borders.

4. Method of a claim 1, comprising the taking of dual or multiple channel images of stained cells.

5. Method of claim 1, further comprising a segmentation of surface stained cells in images.

6. Method of claim 1, further comprising the use of a ridge enhancing curvature depending filter.

7. Method claim 1, comprising ridge enhancement and morphological operators as filling and watershed segmentation.

8. Method of claim 1, comprising the use of adaptive thresholding on ridge enhanced images.

9. Method according to claim 1, wherein organelle transport between cells is investigated.

10. Method according to claim 1, wherein semen quality is investigated.

11. Method according to claim 1, wherein the substrate has been coated to obtain a microarray of essentially singularised cells having predetermined distances to each other.

12. Method according to claim 10, wherein the coating has been applied to the substrate by lithography or photolithography.

13. Method according to claim 1, wherein a chemical compound, a therapeutic substance, a medicament or a suspected pharmaceutically effective substance is added to the culture medium.

14. Method according to claim 1, wherein the cells in the culture medium are subjected to physical effects for a predetermined period.

15. Method according to claim 14, wherein the physical effects are electromagnetic fields.

16. Method according to claim 14 or 15, wherein the physical effects are generated by a therapeutic device.

17. Microscope set-up, comprising a 3-D-microscope, a Z-stepper, and an image acquisition and analysis system for automated cell analysis, cell classification and/or determination of transport and communication between cells in accordance with claim 1.

18. Microscope set-up as claimed in claim 17, further comprising a substrate having a micropatterned coating for obtaining an array of cells having essentially uniform distances to each other.

19. Use of the device according to claim 17 or 18 for serial investigation of the quality of semen.

20. Use of the device of claim 17 to 18 for serial investigation of suspected pharmaceuticals and active mediums.

21. Use of the device of claim 17 or 18 for serial investigation of suspected active substances and active mediums for the treatment of tumours, of high blood pressure, of viral, bacterial or parasitic infection diseases, disorders of the metabolism, disorders of the nervous system, the psyche or the mind, and of the cholesterol level.

22. Use of the device of claim 17 or claim 18 for the investigation of effective substances in gene therapy, for cell targeting and in pharmacology.

23. Pharmaceutical composition which contains a new active substance determined in accordance with claim 1.
